(19) United States
(12) Patent Application Publication
(10) Pub. No.: US 2024/0382183 A1
(43) Pub. Date: Nov. 21, 2024

(54) RAPID SAMPLING OF INTERSTITIAL FLUID USING A MICRONEEDLE ARRAY AND VACCUM-ASSISTED SKIN PATCH

(71) Applicant: William Marsh Rice University, Houston, TX (US)

(72) Inventors: Peter B. LILLEHOJ, Houston, TX (US); Elizabeth Clarke WILKIRSON, Houston, TX (US); Xue JIANG, Houston, TX (US)

(73) Assignee: William Marsh Rice University, Houston, TX (US)

(21) Appl. No.: 18/665,034

(22) Filed: May 15, 2024

Related U.S. Application Data
(60) Provisional application No. 63/466,591, filed on May 15, 2023.

(51) Int. Cl. A61B 10/00

(52) U.S. Cl. CPC A61B 10/0045; A61B 2010/008

(57) ABSTRACT

The present disclosure is directed to devices and methods for the rapid obtention of significant volumes of interstitial fluid from skin. The disclosure describes a microneedle array-based technique capable of collecting up to ˜65 microliters of interstitial fluid within 25 minutes using disposable plastic microneedles in an array, a plastic skin patch, and employing a low-cost manual vacuum pump. Such rapid methods of obtaining these significant quantities of interstitial fluid permits practical application of diagnostic methods using this material.

### PRIORITY CLAIM

This application claims benefit of priority to U.S. Provisional Application Ser. No. 63/466,591, filed May 15, 2023, the entire contents of which are hereby incorporated by reference.

### BACKGROUND

### 1. Field of the Disclosure

The present disclosure relates generally to the fields of medicine, medical devices, and diagnostics. More particular, the disclosure relates to the use of a microneedle (MN) array, skin patch and suction to rapidly obtain significant quantities of interstitial fluid (ISF) from skin (i.e., dermal ISF), in particular amounts useful for determining biomarker content in such fluid and the application in diagnostic methods.

### 2. Background

The detection and quantification of biomolecules in bodily fluids plays an important role in medicine. Currently, the diagnosis and monitoring of many diseases relies on the analysis of blood for the presence of biomolecular markers. While blood sampling is a routine medical procedure, it poses risks of infection (Bogers et al., 2015) and can lead to complications in infants and individuals with blood clotting disorders (Lassandro et al., 2021). Furthermore, the pain associated with blood sampling can deter individuals with needle or blood phobias from getting tested (Bogers et al., 2015). Urine and saliva are less invasive and easier to collect, however, these fluids contain only subsets of the biomarkers found in blood (Piorino et al., 2022), typically at significantly lower concentrations (Sim et al., 2022), hindering their use for many diagnostic applications (Dutkiewicz & Urban, 2016).

ISF is a fluid that surrounds cells and tissues and accounts for 15-25% of the total human body weight (Aukland & Nicolaysen, 1981). ISF is most abundantly found in the lower viable epidermis and the upper dermis (Mccrudden et al., 2015; Samant & Prausnitz, 2018), which is comprised of ISF by up to 70% by volume (Aukland & Nicolaysen, 1981). Prior studies have shown that dermal ISF contains many of the same biomolecules, including metabolites, proteins, and nucleic acids, as blood (Samant & Prausnitz, 2018; Tran et al., 2018; Müller et al., 2012; Kool et al., 2007; Ribet et al., 2023; Miller et al., 2018). For example, glucose has been detected in dermal ISF and its concentration was shown to be highly correlated with concentrations in blood plasma and serum (Samant & Prausnitz, 2018; Jina et al., 2014). Additionally, the pharmacodynamics of glucose in children and young adults and the pharmacokinetics of caffeine in healthy adults were shown to be similar in human ISF and plasma (Samant et al., 2020; Ribet et al., 2020). In addition to biomarkers associated with systemic physiology, dermal ISF contains local biomarkers associated with skin and tissue physiology that are not found in blood (Samant et al., 2020), making it potentially useful for the diagnosis of skin conditions and disorders.

While dermal ISF is a promising source of molecular biomarkers, its use for diagnostic testing is hampered by the lack of rapid and simple techniques for collecting abundant amounts of fluid (Friedel et al., 2023a; Saifullah & Faraji Rad, 2023). Various methods for extracting ISF from skin, including microdialysis (Krogstad et al., 1996), open-flow microperfusion (Pieber et al., 2008), laser microporation (Venugopal et al., 2008), or reverse iontophoresis (Sieg et al., 2004), have been reported; however, they are invasive, time-consuming (˜1 hr), require specialized equipment and need to be performed by trained medical professionals (Kolluru et al., 2019). One commonly used approach for collecting ISF involves the creation of suction blisters to draw fluid to the skin, which is subsequently collected using a hypodermic needle and syringe (Müller et al., 2012; Kool et al., 2007; Samant et al., 2020). While effective, this method requires at least one hr for blistering to occur and can cause prolonged skin erythema and dehydration at the sampling site (Müller et al., 2012). Furthermore, ISF obtained via suction blister contains biomarkers associated with tissue injury, making it less representative of physiologic ISF (Kool et al., 2007).

An alternative strategy for sampling ISF uses MNs to penetrate the skin providing access to ISF in the upper dermis. Compared to hypodermic needles, MNs avoid the nerves and vascular structures located in the deeper layers of the dermis (starting at ˜1,500 m below the skin surface), thereby significantly minimizing their associated pain and risks of infection (Waghule et al., 2019). MNs have been extensively studied for minimally invasive transdermal drug and vaccine delivery, but less research has been reported on using them to extract ISF in humans (Xu et al., 2021). Kasasbeh et al. reported the use of hydrogel-based MNs to extract ISF from human skin, however, this approach required 6 hr of MN application and involved time-consuming and tedious procedures to extract fluid from the MN array (Al-Kasasbeh et al., 2020). Mukerjee et al. demonstrated the extraction of ISF from human skin using a microfluidic device consisting of a hollow MN array connected to a series of microchannels (Mukerjee et al., 2004). For proof of concept, this device was applied to the author's earlobe for 15-20 min, resulting in the extraction of a small droplet (˜50 m in diameter) of ISF. In another study, a hypodermic-based MN device was used to extract 1.1 μL of ISF in 5 min from the forearm (Ribet et al., 2023). Studies by Samant et al. have demonstrated the collection of dermal ISF from human skin using solid metal MNs, which yielded volumes between ˜1-6 μL (Samant et al., 2018; Samant et al., 2020). While these techniques are capable of extracting ISF from human skin, the collected volumes are too low for biomolecular analysis using conventional diagnostic assays, such as enzyme-linked immunosorbent assay (ELISA), Western Blot or lateral flow immunochromatographic assay (LFIA). Miller et al. reported a method for sampling ISF from human skin using hollow MNs which could extract up to 16 μL of ISF, however, this approach required several hours and continual re-application (every 30 min) of the MNs (Miller et al., 2018). Thus, improved methods for assessing biomolecules in ISF are needed.

### SUMMARY

Thus, in accordance with the present disclosure, there is provided a transdermal microneedle array (TMNA) comprising a first microneedle (MN) array composed of a plurality of solid MNs, such as conical, triangular or pyramidal solid MNs. The MN array may be high density, may comprise about 200 MNs per cm² to about 50 MNs cm². It may comprise about 25 to about 1000 MNs or about 100 to about 400 MNs or about 100 to about 200 MNs. The MNs may be arranged in a 3×3, 5×5, 10×10, 15×15, 20×20 or 30×30 configuration. The MN array may have 400 MNs on 10×10 mm substrate, thus 400 needles per sq cm, or 100 MNs on 7.5×7.5 mm substrate, thus 178 MN per square cm. The MN array may be formed using a 3D-printed master mold, such as one made from polydimethylsiloxane (PDMS), and/or the MN array is made of an epoxy-based photoresist material. The MN array may also be made from a ceramic or metallic material. The MN array may be coated with a biocompatible material, such as chitosan, polyethylene glycol, or parylene. The MNs may be about 250 μm or 300 μm to about 1000 μm, about 450 μm to about 750 μm, or about 600 μm in height. The MN array may be about 7.5 mm×7.5 mm, about 10 mm×10 mm, or about 7.5-10 mm×7.5-10 mm. The MN array may be about 300 μm to about 500 μm, or about 400 μm. The MN array may be a 20×20 array with a needle length of about 450 μm. and 3 rectangles in a row

Also provided is a kit comprising the TMNA as described above and a rigid skin patch with one or more cut out configured to fit the TMNA. Rigidity may be defined generally as minimal or less than 5% deformity under a given vacuum pressure. The skin patch may be fabricated from a plastic substrate coated on at least one side with an adhesive. The skin patch may function to allow controlled deformation of the micropores created by the MNs while under such vacuum pressure. Alternatively, the patch may exhibit a sufficient flexural modulus to maintain patch rigidity, for example, the flexural modulus of PMMA ranges from 2.12 to 3.47 GPJa. The patch may be 1.5 mm in thickness and 42.5 in diameter. Alternatively, the patch comprises four squares in a 2×2 matrix that are each 11×11 mm. Another patch comprises 3 rectangles in a row that are 8×15 mm, 9×30 mm, and 8×15 mm. The skin patch may hold a portable vacuum (i.e., suction) cup with the adhesive connection. The kit may further comprise a lateral flow test strip and a vacuum port in operable connection to the lateral flow test strip. The lateral flow test strip may comprise a membrane, such as a nitrocellulose or cellulose membrane, wherein the lateral flow test strip is connected to the patch by microchannels, such as a channel of about 100-500 μm width, or about 200 μm width. The lateral flow test strip may further comprise an antigen binding agent disposed in or on said nitrocellulose membrane.

In another embodiment, there is provided a method of obtaining ISF from a subject using the kit as described above, the method comprising the steps of:
(a) applying the skin patch to a subject;
(b) contacting the subject with the TMNA at the cut out and removing the TMNA thereafter;
(c) affixing the skin patch to the skin over the TMNA insertion site;
(d) applying suction to the skin at the area of the cut out; and
(e) collecting the ISF removed from the subject by the suction.
   Step (b) may comprise multiple applications of the TMNA to the subject at the cutout, such as 2, 3, 4, 5 or more times. The application or applications of the TMNA may be performed by hand or with a spring-loaded applicator. The skin patch may comprise multiple cut outs, such as 2, 3, 4, 5 or more cut outs. The suction may be generated by attaching a hand operated pump or an automated and/or electric pump, such as one generating about −20 to −60 kPa, or about 44 kPa. The suction may be applied for about 5-30 min, such as about 5, about 10, about 15, about 20, about 25 or about 30 mins. About 10 μl, 15 μl, 20 μl, about 25 μl, about 30 μl, about 35 μl, about 40 μl, about 45 μl, about 50 μl, about 60 μl, or about 65 μl of ISF may be obtained. In particular, The MN array may be a 20×20 array and 3 rectangles in a row with a needle length of about 450 μm, the number of MN applications may be three, and the duration of step (d) may be about 20 mins. The method may further comprise analyzing one or more components of collected ISF, such as by detecting their presence by the lateral flow assay.

Also provide is a method of obtaining ISF from a subject using the kit as described above, the method comprising the steps of:
(a) applying the skin patch to a subject;
(b) contacting the subject with the TMNA at the cut out and removing the TMNA thereafter;
(c) applying suction to the skin at the area of the cut out; and
(d) collecting the ISF removed from the subject by the suction, optionally further comprising analyzing one or more components of collected ISF.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Lateral flow-based patch on CNC (top view).

FIG. 2. Lateral flow-based patch on CNC (with dimensions).

FIG. 3. Lateral flow-based patch on CNC (top view).

FIG. 4. Lateral flow-based patch on CNC (with dimensions).

FIGS. 5A-F. Images of the MN array and conventional needles. (FIG. 5A) Optical micrograph of the 20×20 MN array at 20× magnification. Scale bar, 1,000 μm. (FIG. 5B) Close-up view of the 20×20 MN array at 80× magnification. Scale bar, 100 μm. Optical micrographs of a (FIG. 5C) 32-gauge pen needle, (FIG. 5D) 28-gauge lancet, and (FIG. 5E) 27-gauge hypodermic needle tip at 40× magnification for size comparison to the MNs. Scale bars, 500 μm. (FIG. 5F) Side view of the 20×20 MN array at 40× magnification. Scale bar, 500 μm. Inset shows a close-up view of the MNs at 200× magnification. Scale bar, 100 μm.

FIGS. 6A-E. Skin penetration performance of the MN array. Distinct micropores generated in porcine skin using the (FIG. 6A) 10×10 MN array and (FIG. 6B) 20×20 MN array at 20× and 30× magnification, respectively. Scale bars, 1000 μm. Prior to skin insertion, MNs were coated with blue ink for improved visualization. H&E-stained section of porcine skin penetrated by MNs with length of (FIG. 6C) 450 μm, (FIG. 6D) 600 μm, and (FIG. 6E) 750 μm at 1000× magnification. Scale bars, 25 μm.

FIGS. 7A-H. ISF sampling procedure and representative images of extracted ISF. (FIG. 7A) The skin patch sticker is adhered to the anterior forearm, followed by MN insertion using the MN applicator. (FIG. 7B) The rigid plate is attached to the sticker, followed by the attachment of a vacuum cup. Vacuum pressure is generated in the cup using a hand pump. (FIG. 7C) Vacuum pressure is maintained for 20 min for ISF extraction. (FIG. 7D) The vacuum cup is removed and the extracted ISF is collected using capillary tubes. (FIGS. 7E-H) Extracted ISF on the skin of four volunteers. Scale bars, 10 mm.

FIGS. 8A-B. Influence of MN parameters on ISF collection volume. (FIG. 8A) Volume of ISF collected using needle lengths of 450 μm, 600 μm or 750 μm with two or three MN insertions per application site. Significance for two insertions was determined using one-way ANOVA with Tukey's post hoc (ns=P>0.05), and significance for three insertions was determined by two-way ANOVA with Tukey's post hoc (ns=P>0.05, *P=0.0323, **P=0.0303). (FIG. 8B) Volume of ISF collected using needle lengths of 450 μm, 600 μm or 750 μm configured in 10×10 or 20×20 arrays. Significance for the 10×10 array was determined using Student's t test (ns=P>0.05), and significance for the 20×20 array was determined by one-way ANOVA with Tukey's post hoc (ns=P>0.05, *P=0.0113).

FIGS. 9A-D. Proteomic analysis of dermal ISF. (FIG. 9A) Venn diagram showing the overlap in proteins identified in dermal ISF and blood serum samples obtained from five volunteers analyzed using LC-MS/MS. (FIG. 9B) Volcano plot of all proteins identified in dermal ISF vs. blood serum samples. Statistical significance and differential abundance were determined using a minimum −Log₁₀ P-value of 0.05 and a Log₂-fold change of 1, respectively. Points above the horizontal dashed line represent proteins with statistically significant identifications (P-value>0.05). Points to the left of the left-most vertical dashed line denote abundance ratios of ISF/blood serum <0.5, while points to the right of the right-most dashed line denote abundance ratios of ISF/blood serum >2. Points located in the red shaded region denote proteins that are upregulated in ISF while points located in the green shaded region denote proteins upregulated in blood serum. (FIG. 9C) LFIA test results of paired dermal ISF and blood serum samples obtained from two COVID-19 vaccinees (P1, P2) for the detection of SARS-CoV-2 neutralizing antibodies. (FIG. 9D) Concentration of SARS-CoV-2 neutralizing antibodies in dermal ISF collected from 15 COVID-19 vaccinees (n=15) measured using ELISA. Each bar represents the mean±SD of two measurements.

FIGS. 10A-F. Mechanical strength of MN arrays. Force vs. displacement curves for 10×10 MN arrays with needle lengths of 450 μm (FIG. 10A), 600 μm (FIG. 10B) or 750 μm (FIG. 10C), and 20×20 MN arrays with needle lengths of 450 μm (FIG. 10D) 600 μm (FIG. 10E) or 750 μm (FIG. 10F). Solid lines represent the mean from 3 separate measurements using new MN arrays. Shaded region represents the standard deviation. Horizontal dotted lines represent the force required to penetrate human skin.

FIGS. 11A-F. Compression testing of MN arrays at maximum load. Optical micrographs of 10×10 MN arrays with needle lengths of 450 μm (FIG. 11A), 600 μm (FIG. 11B) or 750 μm (FIG. 11C), and 20×20 MN arrays with needle lengths of 450 μm (FIG. 11D) 600 μm (FIG. 11E) or 750 μm (FIG. 11F). Scale bars, 100 μm.

FIGS. 12A-F. MN integrity after repeated skin penetration. Optical micrographs of 10×10 MN arrays with needle lengths of 450 μm (FIG. 12A), 600 μm (FIG. 12B) or 750 μm (FIG. 12C) after 36 insertions in porcine skin, and 20×20 MN arrays with needle lengths of 450 μm (FIG. 12D) 600 μm (FIG. 12E) or 750 μm (FIG. 12F) after 12 insertions. Scale bars, 1,000 μm. Insets show close-up views of the MNs. Scale bars, 100 μm.

FIGS. 13A-B. Designs of the skin patch sticker. MN array application sites outlined in red for the (FIG. 13A) 10×10 MN array and (FIG. 13B) 20×20 MN array.

FIG. 14. ISF collection volume from each participant. Each dot represents the volume of dermal ISF collected from one sample collection. Bars represent the average collection volume sampled from each participant from two independent sample collections.

FIG. 15. Influence of vacuum duration on ISF collection volume. ISF collection volume obtained using varying durations of applied vacuum. Experiments were performed using 20×20 MN arrays with needle lengths of 450 μm or 600 μm and three MN insertions per application site. Each data point represents the mean±SD obtained from six independent sample collections (n=6). Significance was determined by one-way ANOVA with Tukey's post hoc (**p=0.0028, ****p<0.0001).

FIGS. 16A-E. Self-reported pain levels associated with the ISF sampling procedure. (FIG. 16A) Pain level rating scale. (FIG. 16B) Participants' responses for pain level associated with different number of MN insertions per application site. Statistics acquired by Student's t test (ns=P>0.05). (FIG. 16C) Participants' responses for pain level associated with different MN lengths. Significance was determined by one-way ANOVA with Tukey's post hoc (ns=P>0.05). For comparison purposes, participants also rated their perceived pain levels during a standard venipuncture procedure and standard bandage removal. Participants' responses for comparing pain level between (FIG. 16D) MN insertion vs. venipuncture and (FIG. 16E) removal of the skin patch vs. removal of a standard adhesive bandage. Significance was determined with Student's t test (****p<0.0001). Each bar represents the mean±min/max of responses from participants (n=26).

FIGS. 17A-H. Effects of ISF sampling on the skin. Photographs of the sampling site from one participant before MN insertion (FIG. 17A), immediately after MN insertion (FIG. 17B), 2 hr after MN insertion (FIG. 17C), and 24 hr after MN insertion (FIG. 17D) without vacuum application. Photographs of the sampling site from one participant before ISF sampling (FIG. 17E), immediately after ISF sampling (FIG. 17F), 2 hr after ISF sampling (FIG. 17G), and 24 hr after ISF sampling (FIG. 17H). Scale bars, 10 mm.

FIG. 18. Absolution protein concentration in paired dermal ISF and blood serum samples from five volunteers. Each bar represents the mean±SD of three measurements (n=5 participant triplicates).

FIGS. 19A-B. ISF sampling with and without the skin patch. (FIG. 19A) Without the skin patch, the skin deforms excessively when suction is applied, causing the micropores to close. (FIG. 19B) With the skin patch, the skin is made taut when suction is applied, which induces the opening of the micropores, facilitating ISF extraction.

FIGS. 20A-C. Micropore size as a function of MNs length. The insertion of MNs into a wax-based membrane model resulted in pore sizes of (FIG. 20A) 25±3.67 μm for 450 μm-long MNs, (FIG. 20B) 23.8±4.38 μm for 600 μm-long MNs, and (FIG. 20C) 18.6±4.39 μm for 750 μm-long MNs. Each set of data represents the mean±SD of 5 pores (n=5). Scale bars, 100 μm.

FIGS. 21A-I. MN array fabrication process. (FIG. 21A) MN array master is fabricated using a NanoScribe 3D printer. (FIG. 21B) A 3 mm-thick poly(methyl methacrylate) (PMMA) substrate is attached to the backside of the MN array. (FIGS. 21C-D) PDMS master mold is made via replica molding. (FIG. 21E) SU-8 is dropcasted on the PDMS mold. (FIG. 21F) Mold is centrifuged at 4,000 g for 15 min. (FIG. 21G) Exposure to 365 nm UV light for 3 min. (FIG. 21H) Polymerized SU-8 MN array is removed from PDMS mold. (FIG. 21I) MN array is coated in a layer of parylene.

FIGS. 22A-C. Overview of the skin patch design. (FIG. 22A) Exploded view depicting the individual layers of the patch. Topside (FIG. 22B) and internal (FIG. 22C) views of the assembled patch. Scale bars, 6 mm.

FIG. 23. Anti-tetanus toxoid IgG levels in human blood and ISF. Concentration of anti-tetanus toxoid IgG in blood and dermal ISF sampled from four volunteers measured using a commercial ELISA kit. Each bar represents the mean±standard deviation of two measurements (n=2).

FIGS. 24A-C. Characterization of the skin patch. (FIG. 24A) Sequential still frame images showing the extraction and transport of liquid through the patch (without the bandage tape) in an artificial skin model. Arrows indicate the location(s) of the liquid front. Time stamps (min:s) are in the upper right corner. (FIG. 24B) Test results of ISF samples with increasing concentration of anti-tetanus toxoid IgG antibody. The dashed box indicates the desired protective threshold concentration. (FIG. 24C) Test results of ISF samples spiked with anti-tetanus toxoid IgG, anti-diphtheria toxoid IgG, or anti-Bordetella pertussis toxin IgG, and PBS which was used as a blank control.

FIGS. 25A-C. In situ detection of anti-tetanus toxoid IgG in ISF using the skin patch and associated adverse effects. (FIG. 25A) MN insertion in the skin using a MN applicator (i); attachment of the patch to the skin (ii); application of vacuum pressure using a hand pump, followed by removal of the pump and vacuum incubation (iv). (FIG. 25B) Observation of the test results. Inset shows a close-up view of the test results. (FIG. 25C) Photographs of the forearm of a volunteer before MN insertion (i); immediately following MN insertion (ii); immediately after testing and removal of the skin patch (iii); 5 min after testing (iv); 6 hr after testing (v); 24 hr after testing (vi). Dashed boxes indicate the MN insertion sites.

FIGS. 26A-D. Design of the MN array and characterization of MN penetration in porcine skin. (FIG. 26A) Optical micrograph of the MN array at 20× magnification. Scale bar, 1000 μm. (FIG. 26B) Close-up view of the MNs at 80× magnification. Scale bar, 100 μm. (FIG. 26C) Micropores generated in porcine skin following MN insertion using MNs coated with blue ink. Scale bar, 1000 μm. (FIG. 26D) H&E-stained section of porcine skin penetrated by MNs. Scale bar, 25 μm.

FIG. 27. Vacuum-assisted extraction and transport of dermal ISF through the skin patch. Sequential still frame images showing the extraction and transport of ISF through the patch (without the bandage tape) on a volunteer. Arrows indicate the location of the liquid front. Inset shows a close-up view of the test and control lines. Time stamps (min:s) are in the upper right corners.

FIGS. 28A-D. Overview of ISF sampling procedures on human skin. (FIG. 28A) The stencil is adhered to the anterior forearm, followed by MN insertion using the MN applicator. (FIG. 28B) The PMMA plate is attached to the stencil, followed by the attachment of a vacuum cup. Vacuum pressure is generated in the cup using a hand pump. (FIG. 28C) Vacuum pressure is maintained for 20 min. (FIG. 28D) The vacuum cup is removed and the extracted ISF is collected using capillary tubes.

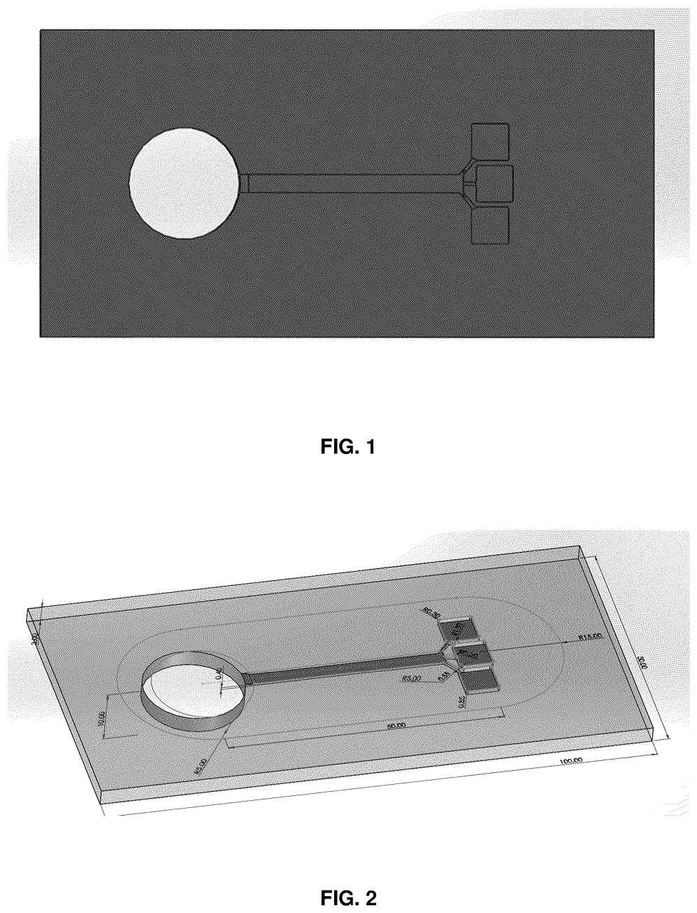

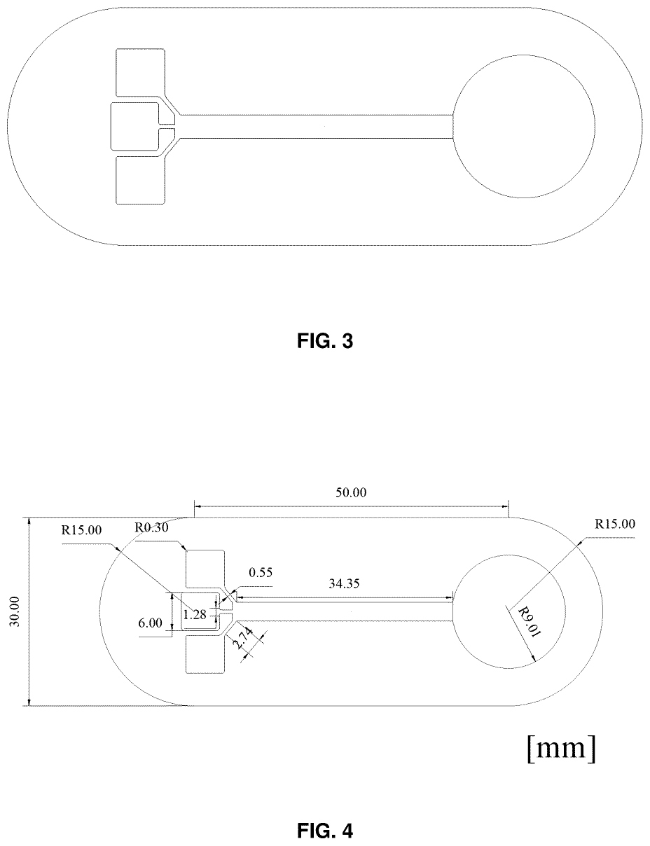

### DESCRIPTION OF ILLUSTRATIVE EMBODIMENTS

As discussed above, recent studies have shown that dermal ISF, a liquid that circulates between cells in bodily tissues, contains many of the same biomolecules (metabolites, proteins, nucleic acids, etc.) as blood, making it a promising source for diagnostic biomarkers. Though there have been a few recent reports of methods for sampling ISF from skin, these methods can only sample small amounts (<10 μL) of ISF while requiring long sampling time (>1 hr), complicated procedures or bulky/expensive instrumentation, resulting in limited value to real world applications.

As described herein, the inventors have developed a sampling technique that is able to collect up to ˜65 μL of interstitial fluid (ISF) within 25 min using a disposable plastic MN array, disposable, plastic skin patch and low-cost manual vacuum pump. Such rapid obtention of significant quantities of ISF opens up the practical application of diagnostic methods using this biofluid. This ISF sampling technique offers several key features resulting in significant improvements over existing sampling methods. First, the technique employs a rigid skin patch that enables micropores to remain stretched and open in a comfortable manner under vacuum pressure, significantly enhancing ISF extraction from skin. When tested on 28 human volunteers, this method yielded an average of 20.7±19.3 μL of fluid within 25 min, which is ˜6-fold more than existing ISF sampling methods reported in literature. Second, unlike existing methods that require specialized equipment or electricity, this approach employs a vacuum cup and hand pump, which are low cost and widely available. And third, the inventors demonstrate that their MN- and vacuum-assisted sampling technique can be integrated with a biosensor on a wearable patch for in situ measurements of protein markers in dermal ISF for point of care testing, which does not require sample processing or handling.

In a particular example, the inventors have developed a simple and minimally invasive technique for rapidly sampling larger quantities of ISF from human skin. In this approach, micropores are generated in the skin using a high-density MN array, followed by the attachment of a rigid skin patch and application of mild vacuum pressure using a portable hand pump. MN arrays of varying sizes and needle lengths were fabricated and characterized to investigate their mechanical strength, skin penetration effectiveness and ISF collection performance. Parameters associated with the sample collection process, including the number of MN insertions and the duration of vacuum application, were studied to optimize the ISF sampling efficiency. Pain levels and skin tolerability were also investigated to assess the safety and acceptability of this technique. Dermal ISF and fingerstick blood collected from human volunteers were analyzed using nano-flow liquid chromatography tandem mass spectrometry (LC-MS/MS) to compare their protein composition and evaluate the diagnostic utility of ISF obtained using this method. Dermal ISF collected from COVID-19 vaccinees was also analyzed for SARS-CoV-2 neutralizing antibodies using two commercially available immunoassays to demonstrate the utility of this approach for ISF-based diagnostic testing.

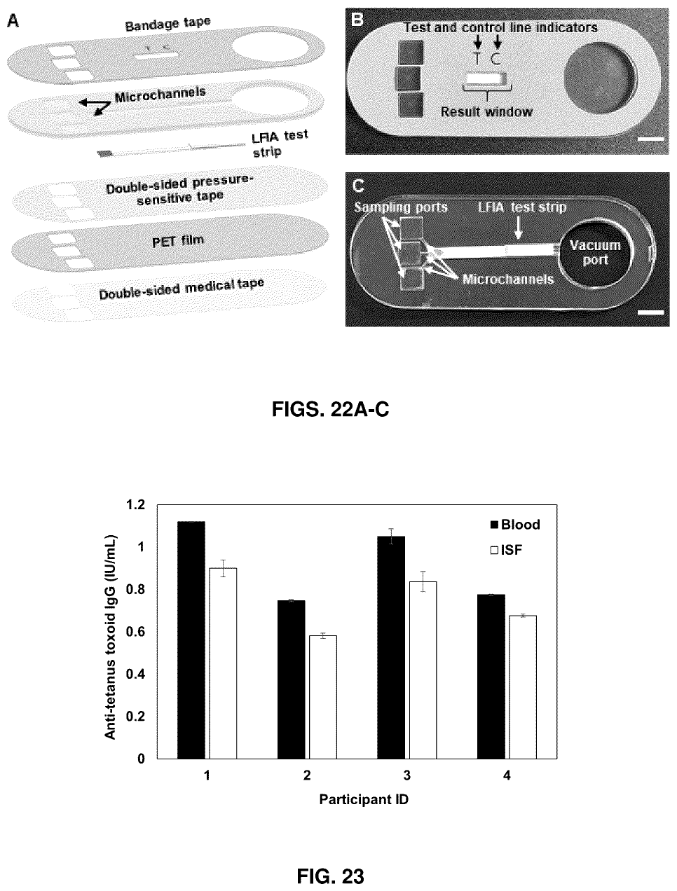

In another aspect, the inventors demonstrate a point-of-care diagnostic test for rapid in situ detection of protein biomarkers in dermal ISF, which offers an instrument-free colorimetric readout that can be interpreted by the naked eye. In this approach, an MN array, such as that shown in FIGS. 22A-B, is used to generate micropores in skin from which ISF is extracted and transported into the skin patch using an integrated, vacuum-assisted extraction system. For proof-of-principle, this device was designed to detect anti-tetanus toxoid IgG, which has clinical relevance in determining an individual's immunity to tetanus infection. Measurements of anti-tetanus toxoid IgG in dermal ISF and blood obtained from four volunteers were performed using a commercial enzyme-linked immunosorbent assay (ELISA) kit to determine the anti-tetanus toxoid IgG levels in both fluids. The functionality of this device was evaluated by testing it on a volunteer, which revealed its ability to accurately detect anti-tetanus toxoid IgG in dermal ISF in <20 min. In addition to its quick turnaround time and ease of use, the only equipment required is a vacuum cup and hand pump (which are inexpensive [<$10] and widely available), making this device well suited for point-of-care diagnostic testing, particularly in resource-limited settings.

These and other aspects of the disclosure are discussed in detail below.

### L. Interstitial Fluid

In cell biology, extracellular fluid (ECF) denotes all body fluid outside the cells of any multicellular organism. Total body water in healthy adults is about 60% (range 45 to 75%) of total body weight; women and the obese typically have a lower percentage than lean men. ECF makes up about one-third of body fluid, the remaining two-thirds are intracellular fluid within cells. The main component of the ECF is the ISF that surrounds cells.

ECF is the internal environment of all multicellular animals, and in those animals with a blood circulatory system, a proportion of this fluid is blood plasma. Plasma and ISF are the two components that make up at least 97% of the ECF. Lymph makes up a small percentage of the ISF. The remaining small portion of the ECF includes the transcellular fluid (about 2.5%). The ECF can also be seen as having two components—plasma and lymph as a delivery system, and ISF for water and solute exchange with the cells.

The ECF, in particular the ISF, constitutes the body's internal environment that bathes all of the cells in the body. The ECF composition is therefore crucial for their normal functions and is maintained by a number of homeostatic mechanisms involving negative feedback. Homeostasis regulates, among others, the pH, sodium, potassium, and calcium concentrations in the ECF. The volume of body fluid, blood glucose, oxygen, and carbon dioxide levels are also tightly homeostatically maintained. The volume of ECF in a young adult male of 70 kg (154 lbs) is 20% of body weight—about fourteen liters. Eleven liters are ISF and the remaining three liters are plasma.

ISF consists of a water solvent containing sugars, salts, fatty acids, amino acids, coenzymes, hormones, neurotransmitters, white blood cells and cell waste-products. This solution accounts for 26% of the water in the human body. The composition of ISF depends upon the exchanges between the cells in the biological tissue and the blood. This means that tissue fluid has a different composition in different tissues and in different areas of the body.

The plasma that filters through the blood capillaries into the ISF does not contain red blood cells or platelets as they are too large to pass through but can contain some white blood cells to help the immune system. Once the ECF collects into small vessels (lymph capillaries) it is considered to be lymph, and the vessels that carry it back to the blood are called lymphatic vessels. The lymphatic system returns protein and excess ISF to the circulation.

The ionic composition of the ISF and blood plasma vary due to the Gibbs-Donnan effect. This causes a slight difference in the concentration of cations and anions between the two fluid compartments.

### II. Microneedles (MNs), Microneedle Patches and Microneedle Arrays (MNAs)

MNs or MN patches or MN arrays are micron-scaled medical devices often used to administer vaccines, drugs, and other therapeutic agents. While MNs were initially explored for transdermal drug delivery applications, their use has been extended for the intraocular, vaginal, transungual, cardiac, vascular, gastrointestinal, and intracochlear delivery of drugs. Many of the beneficial properties that permit these application permit their application here in ISF extraction. MNs are constructed through various methods, usually involving photolithographic processes, or micro-molding. These methods involve etching microscopic structures into resin or silicon to cast MNs. Alternatively, MNs can be fabricated via stereolithography/3D printing. MNs are made from a variety of materials ranging from silicon, titanium, stainless steel, and polymers. Some MNs are made of a drug to be delivered to the body but are shaped into a needle so they will penetrate the skin. The MNs range in size, shape, and function but are all used as an alternative to other delivery methods like the conventional hypodermic needle or other injection apparatus.

MNs are usually applied through even single needle or small arrays. The arrays used are a collection of MNs, ranging from only a few MNs to several hundred, attached to an applicator, sometimes a patch or other solid stamping device. The arrays are applied to the skin of patients and are given time to allow for the effective administration of drugs. MNs are an easier method for physicians as they require less training to apply and because they are not as hazardous as other needles, making the administration of drugs to patients safer and less painful while also avoiding some of the drawbacks of using other forms of drug delivery, such as risk of infection, production of hazardous waste, or cost.

Since their introduction in 1998, several advances have been made in terms of the variety of types of MNs that can be fabricated. The 5 main types of MNs are solid, hollow, coated, dissolvable/dissolving, and hydrogel-forming.

In particular, embodiments, the array comprises 100 needles on 7.5 sq. mm substrate or 400 needles on 10 sq. mm substrate. The patch may be 30-60 mm in diameter, 40-50 mm in diameter, or 43.5 mm in diameter. The patch material may be 1-2 mm thick, such as 1.5 mm thick, and a particular patch material is PMMA. Needles may be solid, conical, and made of SU-8 photoresist coated in parylene.

### II. Vacuum Assist Devices

The vacuum assist devices may be of virtually any kind, including manual (hand, foot or arm driven), electronic, portable, disposable, etc. The vacuum pressure to be attained should be at least about 40 kPa, although such is not necessary for the methods described herein to be operable, and should be maintained at a fixed level for at least about 20 min. Levels above 55 kPa would be disadvantageous due to discomfort to the subject and thus ranges of 25-55, 30-50, and 35-45 kPa are considered useful values for the methods described herein.

### IV. Lateral Flow Devices

Lateral flow immunochromatographic assays (LFIAs), are simple methods/devices intended to detect the presence (or absence) of a target analyte in sample (matrix) without the need for specialized and costly equipment, though many lab-based applications exist that are supported by reading equipment. Typically, these tests are used as low resource medical diagnostics, either for home testing, point of care testing, or laboratory use. Two widely spread and well-known applications are the home pregnancy test and the rapid Covid-19 antigen test.

LFIAs operate generally on the principles of affinity chromatography as the enzyme-linked immunosorbent assay (ELISA). In essence, these tests run the liquid sample along the surface of a pad with reactive molecules that show a visual positive or negative result. The pads are based on a series of capillary beds, such as pieces of porous paper, microstructured polymer, or sintered polymer. Each of these pads has the capacity to transport fluid (e.g., urine, blood, saliva) spontaneously.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (i.e., lines) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the line-area changes color.

Typically, there are at least two lines: one (the control) that captures any particle and thereby shows that reaction conditions and technology worked fine, the second contains a specific capture molecule and only captures those particles onto which an analyte molecule has been immobilized. In this way, the presence of two lines indicates a positive result, whereas the presence of just one line (control) indicates a negative result. After passing these reaction zones, the fluid enters the final porous material—the wick—that simply acts as a waste container. LFIAs can operate as either competitive or sandwich assays.

### IV. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

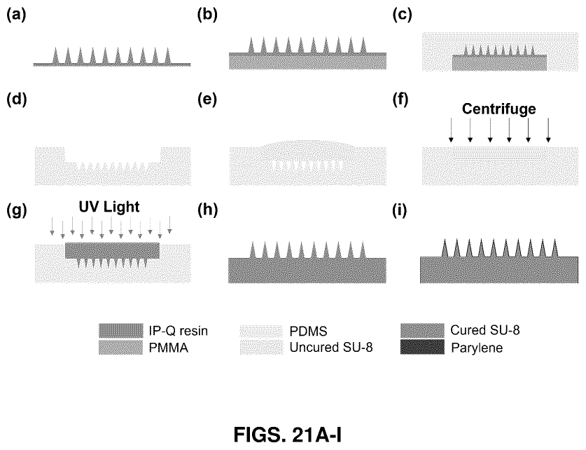

Fabrication of MN arrays. MN arrays were designed using NX software (Siemens, TX, USA) and printed in IP-Q resin using a Photonic Professional GT lithography system (NanoScribe, MA, USA). 3 mm thick PMMA (McMaster Carr, IL, USA) was attached to the backside of the MN array for enhanced rigidity. MN arrays were fabricated via centrifugation-assisted replica molding (FIG. 21A-I). MN array master molds were made from PDMS (Sylgard 184, Dow, MI, USA). The PDMS was mixed at a 1:10 (curing agent-to-elastomer) ratio, degassed for 30 min, poured onto the MN array master and heated in a convection oven at 80° C. for 2 hr. Cured PDMS was cut into individual molds using a razor blade and submerged in 70% isopropanol for 30 min. PDMS molds were dried overnight at room temperature before use. This process was used to create master molds for different sized MN arrays. MN array replicas were fabricated by pouring SU-8 2025 photoresist (Kayaku Advanced Materials, MA, USA) into the PDMS molds followed by centrifugation at 4,000 g for 15 min. The molds were then placed under a 50 W UV (365 nm) lamp for 3 min for SU-8 polymerization. MN arrays were coated with 1.5 m of parylene using a Labcoater 2 parylene deposition system (Specialty Coating Systems, IN, USA). MN arrays were characterized and imaged using a VHX-7000 optical microscope (Keyence Corporation, Osaka, Japan).

Mechanical testing of the MN arrays. The compression strength of the MN arrays was measured using a mechanical testing system (Instron, MA, USA). For each measurement, a single MN array was placed on the bottom plate of a 100 N load cell with the MN tips facing upward. The top plate was compressed from 0 to 90 N at a travel velocity of 0.5 mm min⁻¹. Force-displacement curves were obtained from three different MN arrays for each design, normalized in MATLAB (MathWorks, MA, USA) to set the initial position of the plate at zero displacement and plotted as the mean data±standard deviation (SD) in Microsoft Excel. Optical images of the MN arrays were taken before and after mechanical testing using a Keyence VHX-7000 microscope.

Skin penetration testing. MN arrays were tested on porcine skin to evaluate their skin penetration performance. Cadaver porcine skin from the abdominal with hair, fat and subcutaneous tissue removed was purchased from Animal Technologies, Inc. (TX, USA). The skin was cut into 10 cm×10 cm sections, vacuum sealed, and stored at −20° C. Prior to testing, a frozen skin section was thawed at room temperature and mounted onto foil-wrapped cardboard using safety pins. MNs tips were coated in blue ink using a fine tip paintbrush (Zem Brush MFG, OH, USA), and the MN array was inserted into the skin section using a MN applicator (Micropoint Technologies, Singapore). To evaluate the durability of the MNs after repeated skin insertion, MN arrays were inserted into porcine skin 12 (20×20 array) or 36 (10×10 array) times, which were the maximum number of MN insertions to generate micropores for ISF extraction, using the MN applicator. Optical images of the MN arrays immediately following MN insertion (without post-cleaning) were obtained using a Keyence VHX-7000 microscope. To evaluate the pore size generated from the MNs, MN arrays were inserted into a flexible, wax membrane (comprised of 8 layers of Parafilm M) using the MN applicator. The Parafilm membrane was imaged using a Keyence VHX-7000 microscope and pore size measurements were performed using the VHX-7000 microscope software (Ver 1.4.14.169). Results were presented as the average±SD from 5 measurements for each MN length. To visualize the MN insertion wounds, histological analysis was performed on porcine skin sections following MN insertion. MNs were coated with Trypan blue (Sigma-Aldrich, USA) in glycerol (Sigma-Aldrich) solution using a fine tip paintbrush, and the MN array was inserted into the skin section using a MN applicator. The skin sample was fixed in a 10% formalin solution (Sigma-Aldrich) for at least 48 hr, transferred and stored in a 70% ethanol solution. The sample was then embedded in paraffin (Sigma-Aldrich), dehydrated, sectioned, and stained with hematoxylin and eosin (H&E). Optical images of H&E-stained skin sections were captured using a Keyence VHX-7000 microscope.

Fabrication of the skin patch. The skin patch consists of a sticker and rigid plastic plate, both containing rectangular cutouts for the MN insertion sites. The sticker was fabricated from medical grade, double-sided adhesive tape (3M Company, MN, USA) and the rigid plate was fabricated from 1.5 mm thick PMMA (McMaster Carr). Double-sided, pressure-sensitive adhesive tape (Adhesives Research Inc., PA, USA) was attached to the top side of the plate. The sticker and rigid plate were designed using AutoCAD software (Autodesk, CA, USA) and cut using a CO₂ laser cutter (Universal Laser System, Inc., AZ, USA). The interior edges of the plate were sanded using a Dremel rotary tool to create smooth points of contact with the skin.

Sample collection from human volunteers. All procedures involving humans were conducted under the guidance and approval from the Rice University Institutional Review Board (IRB-FY2021-147). Criteria for participation was as follows: adults or Rice University students ages 18 or older with no blood clotting disorders (including hemophilia, or factor II, V, VII, X, or XII deficiencies) or known skin allergies to medical adhesives. Potential participants were provided with informed consent to participate in the study. Participants were explained the entirety of the sample collection process prior to beginning the study. Informed consent of all participating subjects was obtained.

Twenty-eight adults were recruited for the study. ISF collection was carried out by first cleaning the participant's forearm using an alcohol prep pad (Fisher Healthcare, MA, USA) and attaching the skin patch sticker. The MN array was then applied to the skin two or three times at the MN insertion sites using a MN applicator. Immediately following MN insertion, the plastic plate was attached to the sticker followed by the attachment of a vacuum cup (Hansol Medical, South Korea). After ˜3 min, vacuum pressure (˜44 kPa) was generated inside the cup using a hand pump (Hansol Medical) and maintained for 20 min. The vacuum cup was then removed and the extracted ISF was collected using capillary tubes (Thermo Fisher Scientific, MA, USA and Drummond Scientific Company, PA, USA). The skin patch was gently removed from the skin using an adhesive remover pad (Torbot Group, RI, USA) and the sampling site was cleaned using a fresh alcohol prep pad. After the study, participants were asked to complete a questionnaire rating the perceived pain levels associated with different steps of the sample collection procedure. The collected ISF sample was transferred to a low-bind microcentrifuge tube (Eppendorf, Hamburg, Germany), incubated at room temperature for 1 hr, and centrifuged at 10,000 g for 10 min. The supernatant was transferred to a new low-bind microcentrifuge tube, snap frozen in liquid N₂ for 5 min, and stored at −80° C. until analysis.

Blood samples were obtained via fingerstick using a lancing device (Bayer Microlet) and 30G lancets (CareTouch). Blood was collected in capillary tubes (Thermo Fisher Scientific), transferred to a low-bind microcentrifuge tube, and incubated for 1 hr at room temperature. The tube was then centrifuged at 10,000 g for 10 min. Separated serum was transferred to a new low-bind microcentrifuge tube, snap frozen in liquid N₂ for 5 min, and stored at −80° C. until analysis.

Proteomic analysis. Dermal 1SF and plasma samples were first processed using a High Select™ Depletion Spin Column (Thermo Fisher Scientific, A36369) to remove abundant proteins. Samples were prepared for LC-MS/MS analysis by adjusting the sample solution to a final concentration of 5% sodium dodecyl sulfate (SDS), tetraethylammonium bromide (TEAB, 50 mM, pH 7.55, 25 μL). The samples were then centrifuged at 17,000 g for 10 min to remove debris. The supernatant was transferred to a clean tube and proteins were reduced by making TCEP (20 mM, Thermo Fisher Scientific, 77720) and incubated at 65° C. for 30 min. The sample was cooled to room temperature and iodoacetamide acid (0.5 M, 1 μL) was added and allowed to react for 20 min in the dark. Next, phosphoric acid (12%, 2.75 μL) was added to the protein solution, and binding buffer (90% methanol, 100 mM TEAB, final pH 7.1, 165 μL) was then added to the solution. The resulting solution was added to a S-Trap spin column (Protifi, Fairport, NY) and passed through the column using a benchtop centrifuge (30 sec spin at 4,000 g). The spin column was washed with 400 μL of binding buffer (90% methanol, 100 mM TEAB, pH 7.55) and centrifuged. This process was repeated 2 more times. Trypsin was added to the protein mixture at a ratio of 1:25 in TEAB (50 mM, pH 8) and incubated at 37° C. for 4 hr. Peptides were eluted with TEAB (50 mM, 80 μL) followed by formic acid (0.2%, 80 μL) and finally acetonitrile (50%, 80 μL). The combined peptide solution was then dried in a SpeedVac and resuspended in acetonitrile (2%), formic acid (0.1%), water (97.9%) and placed in an autosampler vial.

Peptide mixtures were analyzed by LC-MS/MS using a nanoflow LC chromatography system (UltiMate 3000 RSLCnano, Thermo Scientific, San Jose, CA), coupled on-line to a Thermo Orbitrap Eclipse mass spectrometer (Thermo Fisher Scientific) through a nanospray ion source coupled with a high field asymmetric waveform ion mobility spectrometry (FAIMS) Pro device (Thermo Fisher Scientific) with Instrument Control Software (version 3.4). FAIMS separations were performed at standard resolution with the following settings: inner and outer electrode temperature=100° C.; FAIMS gas flow=0 L min⁻¹, Compensation Voltages (CV): −35, −55 and −75 with 1.3 sec cycle times per CV. A direct injection method was used. MS1 mass spectra were acquired using a resolution setting of 120,000 (at 200 m z⁻¹), scanning from 400-1600 m z⁻¹. Peptides were selected for MS/MS by data-dependent acquisition. Selected peptides were fragmented using higher energy collisional dissociation (HCD) with a setting of 30% normalized collision energy and peptide fragments were detected in the Orbitrap Eclipse ion trap using a Turbo scan rate. The analytical column was an Aurora capillary LC column (75 m×25 cm, 1.6 μm) obtained from Ion Opticks (Fitzroy, Vic, Australia). After equilibrating the column in 97% solvent A (0.1% formic acid in water) and 3% solvent B (0.1% formic acid in acetonitrile), the samples (2 μL in solvent A) were injected at 450 nL min⁻¹ for 5 min when the flow was lowered to 300 nL min⁻¹. Peptides were eluted from the C18 column using a mobile phase gradient as follows: 3% to 6% 5-5.1 min, 6% to 26% B, 5.1-125 min; 26% to 40% B, 125-137 min; 40% to 90% B, 137-140 min; isocratic at 90% B, 140-141 min; 90% to 5%, 141-142 at 450 nL min⁻¹; isocratic at 5% 142-142.5 min; 5% to 95% 142.5-143 min; isocratic at 95% B 143-144 min; 95% to 5% B 144-145 min and isocratic at 3% B until 160 min.

Protein identification. Tandem mass spectra were extracted and charge state deconvoluted by Proteome Discoverer (Thermo Fisher, version 2.5). Deisotoping was not performed. All MS/MS spectra were searched against a Uniprot human database and a common contaminant database (cRAP, version 03-29-2016) using SEQUEST. Searches were performed with a parent ion tolerance of 5 ppm and a fragment ion tolerance of 0.60 Da. Trypsin is specified as the enzyme, allowing for two missed cleavages. Fixed modification of carbamidomethyl © and variable modifications of oxidation (M) and deamidation were specified in SEQUEST. The protein FDR validator node was used to estimate to calculate experimental q-values and a cut-off of 1.0% FDR was applied.

Proteomic identification results were further analyzed using two online biomarker databases, OncoMX⁴⁸ and BIONDA.⁴⁹ In each database, the accession number was entered and the results were filtered with a Python code. In OncoMX, data were reported as an FDA or an EDRN biomarker. In BIONDA, data were reported as the associated disease. All results were manually cross-checked for accuracy.

Absolute protein concentration measurements. Absolute concentrations of protein in blood and ISF samples were determined using a Pierce Coomassie (Bradford) Protein Assay Kit (Thermo Fisher Scientific). Paired dermal ISF and fingerstick blood samples from five volunteers were analyzed in triplicate. The samples were 100× diluted in PBS. Bovine serum albumin (BSA) standards were diluted to 1,500, 1,000, 750, 500, 250, 125, 25, 0 μg mL⁻¹ with PBS. The standard microplate protocol with a working range of 125-1500 μg mL⁻¹ was used. Briefly, each standard or sample (5 μL) was mixed with Coomassie reagent (250 μL) in a microplate well (Thermo Fisher Scientific), then incubated for 10 min at room temperature. Absorbance measurements were read at 595 nm using a Biotek Epoch microplate spectrophotometer (Agilent, CA, USA).

SARS-CoV-2 neutralizing antibody detection in dermal ISF. Paired dermal ISF and fingerstick blood samples from volunteers were analyzed for the presence of SARS-CoV-2 neutralizing antibody using a lateral flow antibody detection device (RayBiotech, USA). Tests were performed according to the manufacturer's instructions using freshly collected fingerstick blood or dermal ISF. Images of the test results were captured using a smartphone camera. SARS-CoV-2 neutralizing antibody concentrations were measured in dermal ISF using a SARS-CoV-2 Surrogate Virus Neutralization test kit (GenScript USA, USA). Briefly, dermal ISF samples and SARS-CoV-2 neutralizing antibody standard (GenScript USA, USA) with concentrations of 0, 9.375, 18.75, 37.5, 75,150,300, 600 ng mL⁻¹ were prepared, and each sample (10 μL) was diluted with sample dilution buffer (90 μL). Diluted samples were mixed with diluted horseradish peroxide (HRP) conjugated recombinant SARS-CoV-2 receptor binding domain (RBD) fragment (HRP-RBD) solution with a 1:1 volume ratio. Each mixture (100 μL) was added to the corresponding well. The plate was covered, incubated at 37° C. for 15 min and wells were rinsed four times with wash buffer. 3,3′,5,5′-Tetramethylbenzidine solution (100 μL) was added to each well and the plate was incubated in the dark at room temperature for 15 min. Stop solution (50 μL) was added to each well to quench the reaction. The colorimetric signal was read immediately using a Biotek Epoch microplate spectrophotometer at a wavelength of 450 nm. Duplicate measurements were run for each sample.

Statistics. Statistical analysis was performed using GraphPad Software (Prism 9.5 version). Statistical differences were determined using a two-tailed Student's t test, one-way ANOVA with Tukey's post hoc, or two-way ANOVA with Tukey's post hoc, according to the number of groups being analyzed. The type of test was indicated in conjunction with each P-value when reported throughout the manuscript. P<0.05 was considered statistically significant in all cases.

### Example 2—Results

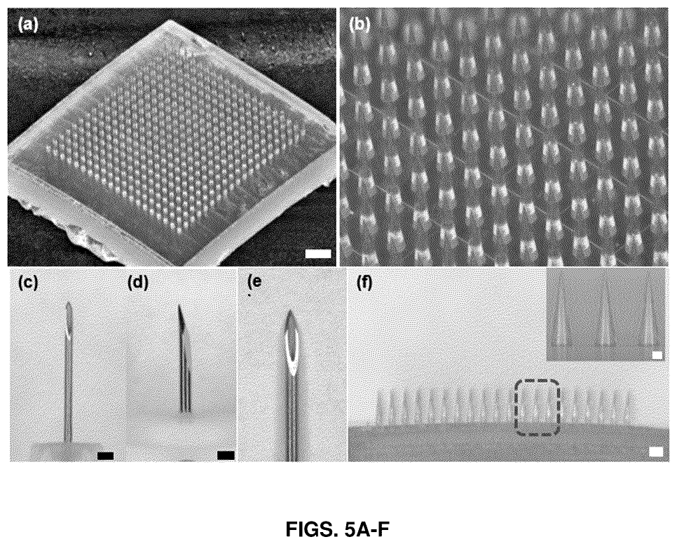

Design and characterization of the MN array. The MN arrays are comprised of solid, conical MNs made from polymerized SU-8 photoresist (FIGS. 5a-b). While polymerized SU-8 is a biocompatible material with low cytotoxicity and minimal reaction in tissue (Chen & Lee, 2021), MNs were coated with 1.5 m of parylene to further enhance their biocompatibility (Kuppusami & Oskouei, 2015). MNs were designed to safely penetrate human skin multiple times to create thousands of micropores while maintaining a compact profile to minimize discomfort. MN arrays with three different needle heights (450, 600 and 750 μm) were fabricated to determine the optimal length for extracting the greatest amount of ISF with the least amount of pain. A base diameter of 200 m was used for all the MNs, making their overall size much smaller than conventional needles (FIGS. 5c-e). MNs were configured in a two-dimensional array (10×10 or 20×20) to multiply the number of micropores generated per insertion, with a needle-to-needle spacing of 400 m for each size array (FIG. 5f). The overall size of the 10×10 and 20×20 arrays were 7.5×7.5 mm and 10×10 mm, respectively.

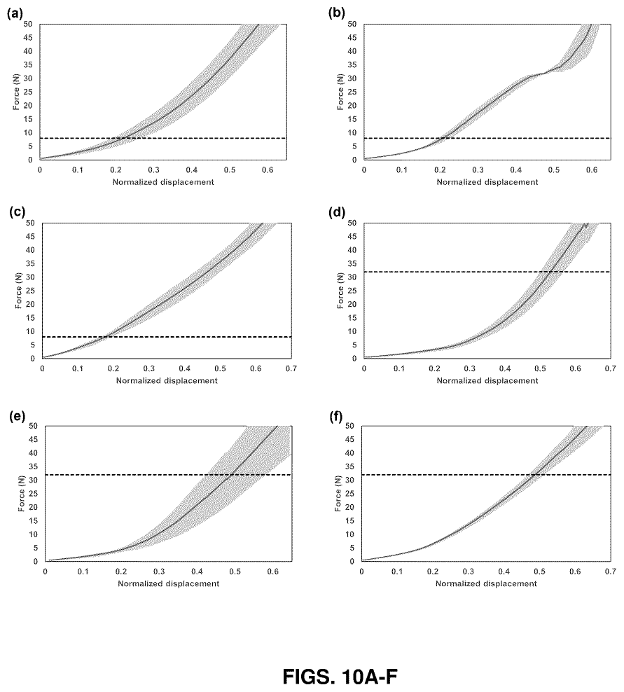

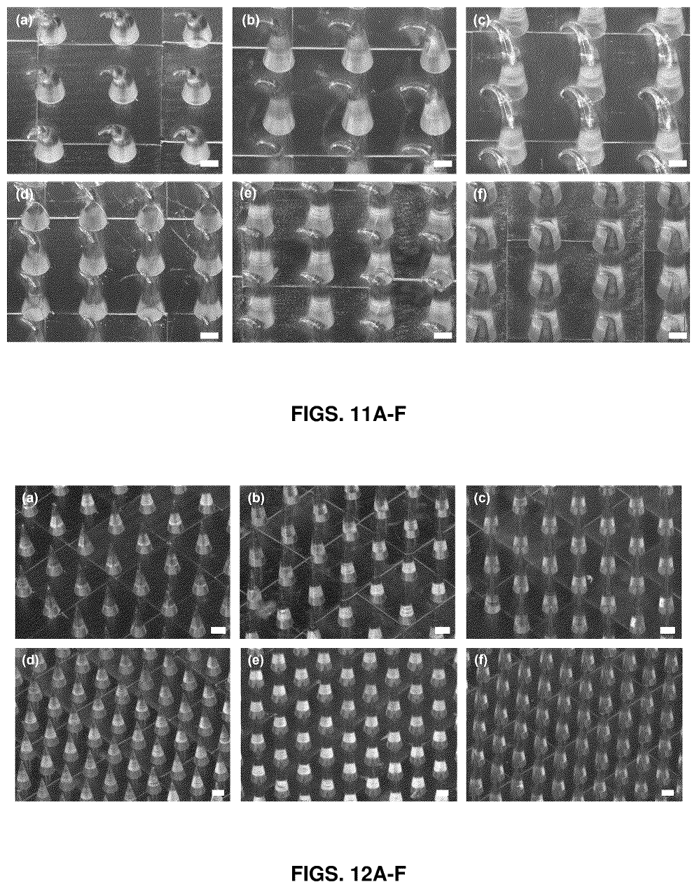

The mechanical strength of the MN arrays was characterized to assess their ability to safely penetrate human skin. Force-displacement curves were generated of 10×10 and 20×20 MN arrays with needle lengths of 450 μm, 600 μm and 750 μm subjected to mechanical compression (FIGS. 10A-F). The 10×10 and 20×20 MN arrays did not exhibit any signs of deformation when compressed up to 50 N, which is at least 1.5-fold larger than the force required to penetrate human skin (0.08 N per MN) (Davis et al., 2004). Subjecting the MN arrays to compression>50 N caused the tips of the MNs to undergo plastic deformation (FIGS. 11A-F); however, none of the MNs exhibited signs of failure (i.e., fracture). These results indicate that the MNs can penetrate human skin and will not break during skin insertion, thereby eliminating potential complications associated with MN failure.

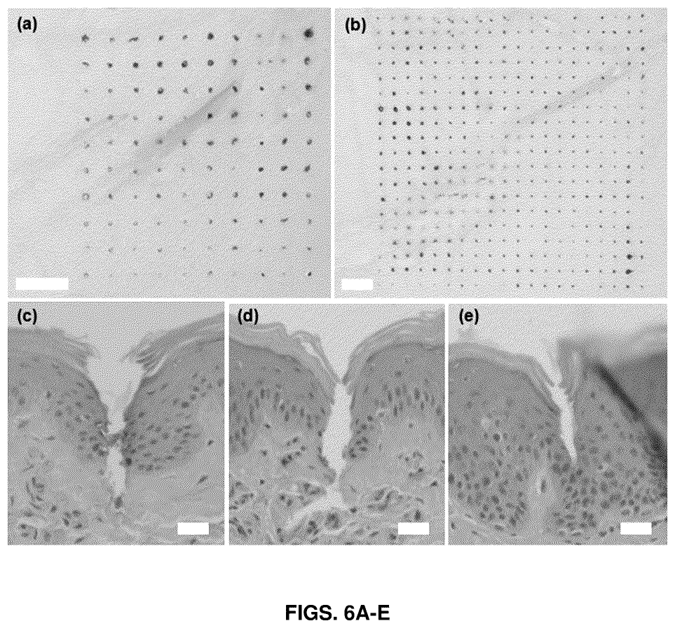

The inventors assessed the capability of the MNs to generate micropores in skin by applying the MN arrays to porcine skin, which was used as an anatomically and biochemically similar model as human skin (Schmook et al., 2001). Prior to skin insertion, MNs were coated with blue ink for improved visualization. Distinct micropores were generated by each MN, which were confined to the needle penetration sites with no impact to the surrounding tissue (FIGS. 6a-b). Histological analysis was also performed to evaluate the effects of MN penetration in skin tissue. Each MN insertion site was characterized by a conical micropore that pierces through the epidermis (FIGS. 6c-e). The formation of these cavities provides access to dermal ISF in the upper dermis, while avoiding the dense collection of nerves and vascular structures located in the lower dermis layer. MN integrity was evaluated by applying the MN array to skin multiple times. Skin samples were punctured 36 times using the 10×10 MN arrays and 12 times using the 20×20 MN arrays, which are the maximum number of times that the MNs are inserted into the skin to generate micropores for ISF extraction. Optical micrographs of the MN array following repeated skin insertion revealed that the MNs exhibited no discernable deformation or damage (FIGS. 12A-F).

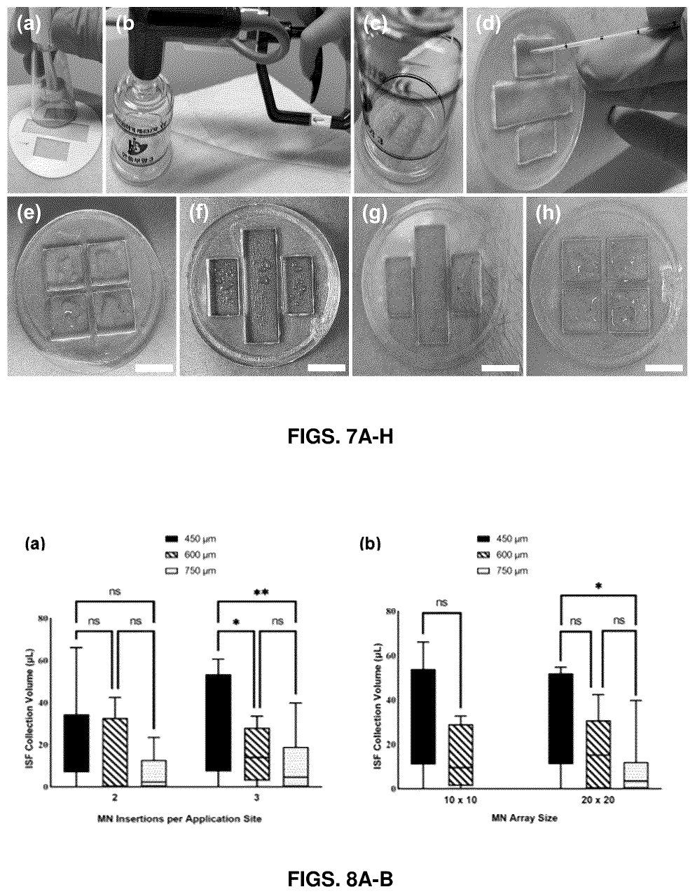

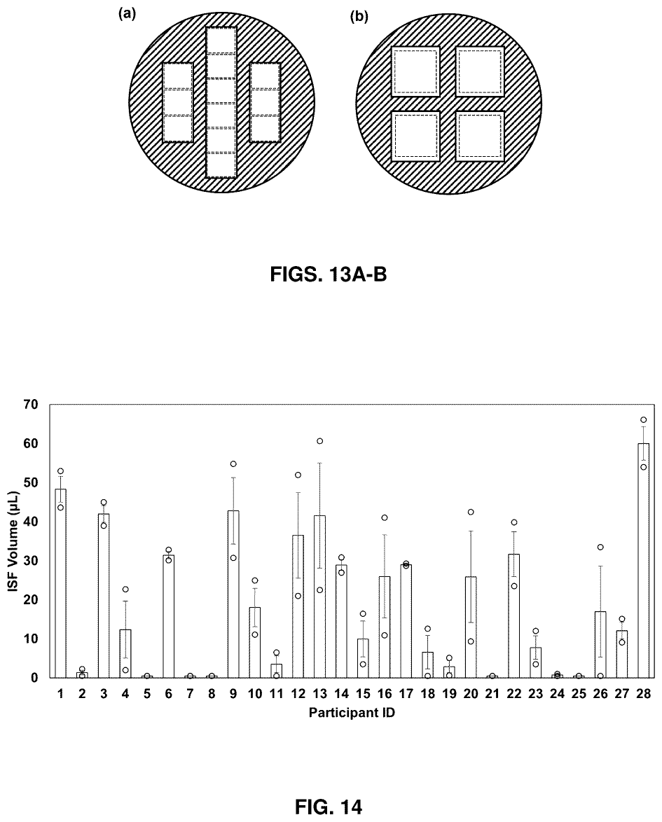

Vacuum-assisted sampling of ISF from human skin using the skin patch. ISF was collected from 28 adults at Rice University, whose demographics are listed in Table S1. The ISF sampling procedure is shown in FIGS. 7a-d. A double-sided skin-friendly adhesive sticker was first adhered to the anterior forearm, which was selected as the ISF sampling site due to its ease of access and lack of excess body hair. The sticker contains rectangular cutouts, which serve as guides for the MN insertion sites. For the 10×10 MN array, the cutouts are arranged in three columns, resulting in 12 distinct MN insertion sites (FIG. 13a). For the 20×20 MN array, the cutouts are arranged as a 2×2 matrix, resulting in four distinct MN insertion sites (FIG. 13b). The MN array was applied to the skin two or three times at each insertion site using a spring-loaded applicator (FIG. 7a), which generated thousands of micropores in the skin in a rapid and repeatable manner. A rigid plastic plate (containing identical cutouts as the sticker) was adhered to the sticker, followed by the attachment of a vacuum cup. Vacuum pressure (˜44 kPa) was generated inside the cup using a hand pump (FIG. 7b), resulting in pressure-driven convection of ISF through the micropores. The skin patch kept the skin taunt when vacuum pressure was applied, which induced the opening of the micropores for increased ISF extraction. The extracted ISF was pooled on the skin forming small droplets, which were confined within the rectangular wells of the patch (FIG. 7c). After 20 min, the vacuum cup was removed and the ISF was collected using capillary tubes (FIG. 7d).

TABLE S1
Demographics of the 28 participants
enrolled in the ISF collection study.
Age (years)

| | |
|---|---|
| Mean ± SD | 24.1 ± 3.8 |
| Median | 23.5 |

Sex

| | |
|---|---|
| Male | 13 |
| Female | 15 |

Ethnicity

| | |
|---|---|
| Caucasian | 7 |
| African American | 2 |
| Hispanic | 2 |
| Asian | 14 |
| Multiple | 3 |

ISF collected using this technique was clear to light yellowish in color and generally more viscous than sweat (FIGS. 7e-h), which is consistent with prior observations of ISF extracted from human skin (Samant et al., 2020). Precautions were taken during the sampling procedure to ensure that the ISF sample was not contaminated with sweat. Sample collection was performed in a temperature—(68-72° F.) and humidity—(˜40-50% RH) controlled environment to minimize perspiration. Prior to sample collection, the participant's skin was cleaned with an alcohol prep pad and thoroughly dried, further reducing the likelihood of sample contamination with sweat or environmental residues. The inventors investigated whether the ISF sampling procedure could cause the secretion of sweat by adhering the skin patch to the forearm of a volunteer and applying suction for 20 min using the vacuum cup (without MN insertion). No fluid was observed on the skin, confirming that the ISF sample was not contaminated with sweat. Microscopic inspection of collected ISF revealed the absence of red blood cells, which are markers for whole blood contamination (Youl et al., 2005), thus confirming that the ISF samples were not contaminated with blood.

The average amount of ISF collected from all 28 participants was 20.8±19.4 μL (mean±SD) where up to 66.1 μL was collected from one participant. The amount of ISF collected from each participant is presented in FIG. 14. Variability in the amount of dermal ISF collected from different individuals using the same MN parameters and sample collection procedure was observed. The inventors attribute this variability to differences in the participants' skin, such as topology, thickness, elasticity, and hydration, which can affect the MN penetration depth and micropore size. Intrasubject and intersubject variability in the sample collection volume was also reported by Samant et al. using a MN- and vacuum-assisted technique for extracting ISF from skin (Samant et al., 2020). Furthermore, prior studies have shown variability in the collection of other bodily fluids, including blood (Grady et al., 2014), sweat (Baker, 2017), and saliva (Ghezzi et al., 2000), among different individuals.

The inventors investigated the influence of several MN parameters, including the array size (10×10 and 20×20), needle length (450 μm, 600 μm and 750 μm), and the number of MN insertions (2 or 3) per application site, on the amount of ISF that could be collected from participants (FIGS. 8a-b). They observed that the MN length did not have a significant effect on the ISF collection volume when applied two times per application site. However, the 450 μm-long MNs resulted in a significantly larger amount of ISF than the 600 μm-long and 750 μm-long MNs when applied three times per application site. Additionally, in a 20×20 array format, the 450 μm-long needles resulted in the collection of significantly more ISF than the 750 μm-long MNs. Based on these collective results, using a 20×20 MN array with a needle length of 450 μm and three MN insertions per application site resulted in the greatest amount of collected ISF.

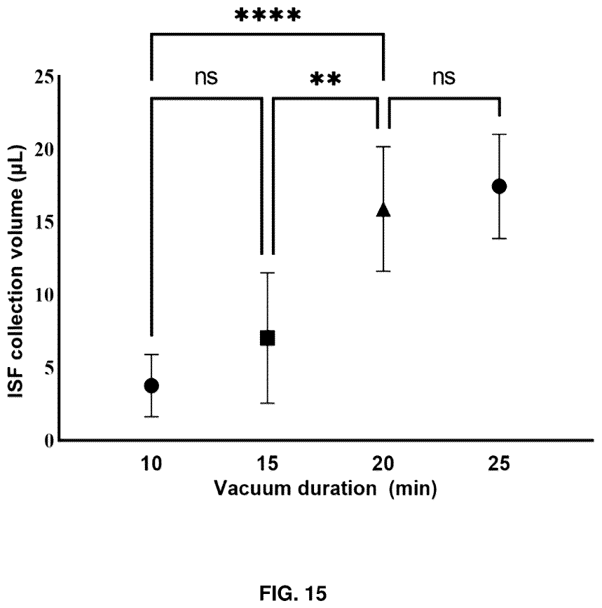

The influence of the vacuum duration on the ISF collection volume was also studied by varying the amount of time that suction was applied to the skin. The inventors observed a positive correlation between the vacuum duration and the ISF collection volume where longer durations of applied vacuum resulted in the extraction of larger amounts of ISF (FIG. 15). There was a significant increase in the amount of ISF collected from participants by applying suction for 20 min compared with 10 or 15 min. However, there was not a significant increase in the ISF volume when suction was applied for >20 min, which is consistent with prior studies showing that ISF extracted from the skin plateaus after ˜20 min (Samant et al., 2020). Furthermore, minor complications, like erythema, edema and ecchymosis, can occur to the skin when suction is applied for >20 min (Rozenfeld & Kalichman, 2016). Therefore, 20 min was selected as the optimal duration for vacuum application following MN insertion.

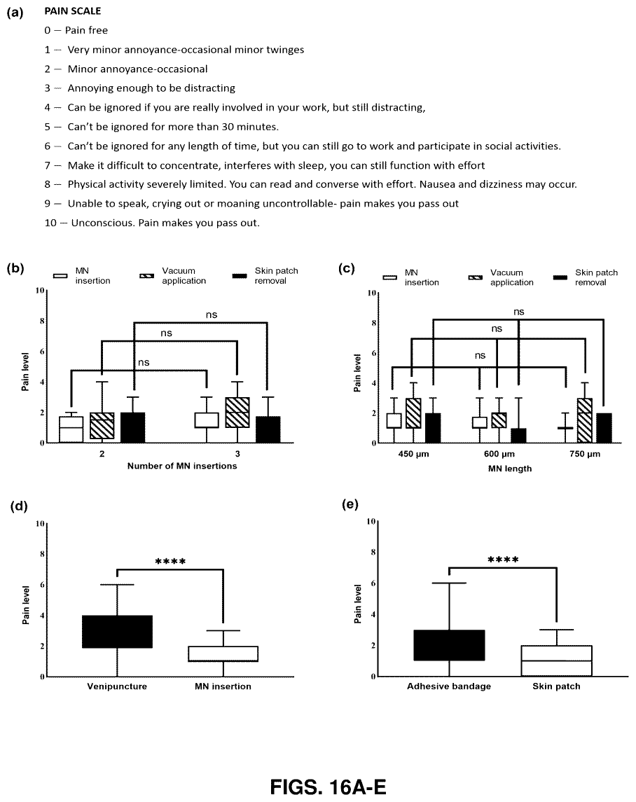

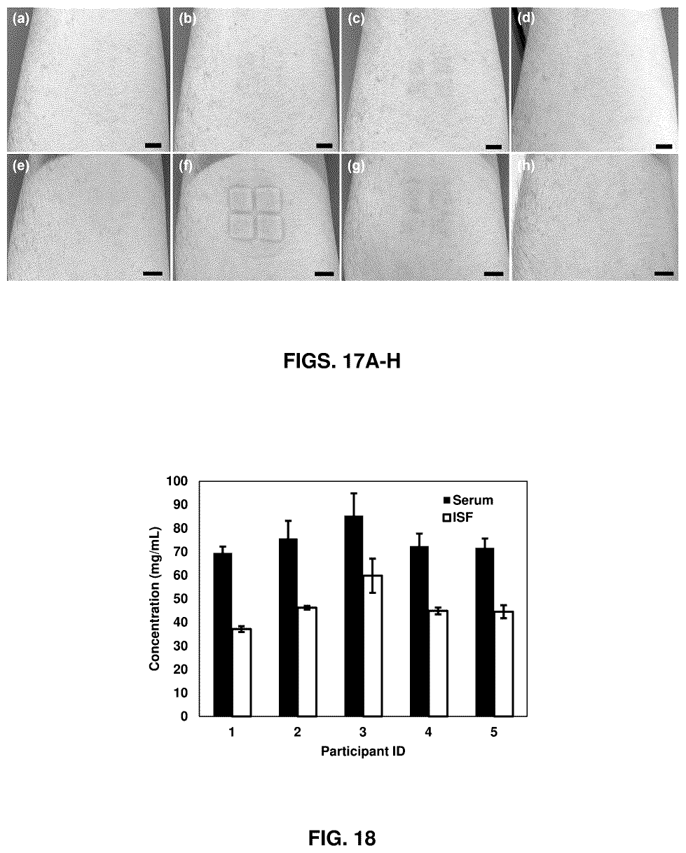

Participants completed a survey to rate the pain level (on a scale of 0 to 10, with 0 being painless and 10 being unbearable, FIG. 16a) associated with the MN insertion, vacuum application and removal of the skin patch (FIGS. 16b-e). The pain level reported for the entire sample collection procedure by all participants was 1.27±1.03 (mean±SD) where 33% of the participants rated the pain level as <1 (pain free). The inventors also investigated whether the ISF sampling procedure caused any adverse effects to the skin. MN insertion resulted in slight skin redness at the MN application sites, which resolved within 24 hr (FIGS. 17a-d). More pronounced skin redness, mild swelling, and slight tenderness localized within the skin patch was observed as a result of suction being applied to the skin (FIGS. 17e-h), however, these reactions are common and benign effects associated with cupping/vacuum therapy (Dalton & Velasquez, 2017). Overall, the collection of ISF using this technique was well tolerated with minor adverse effects that completely resolved within one day.

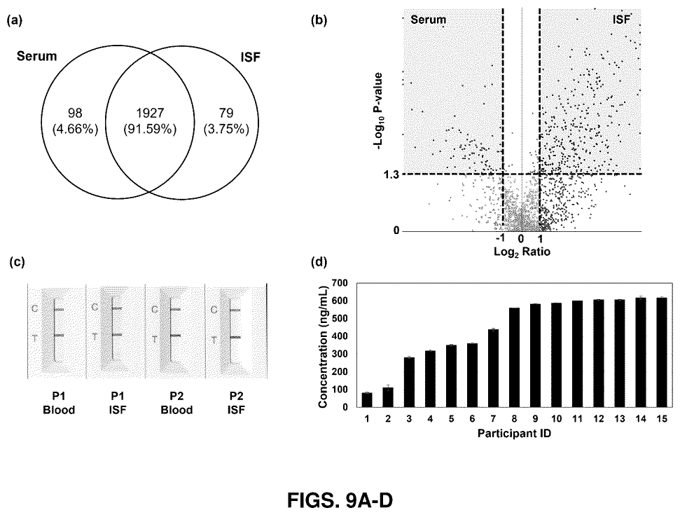

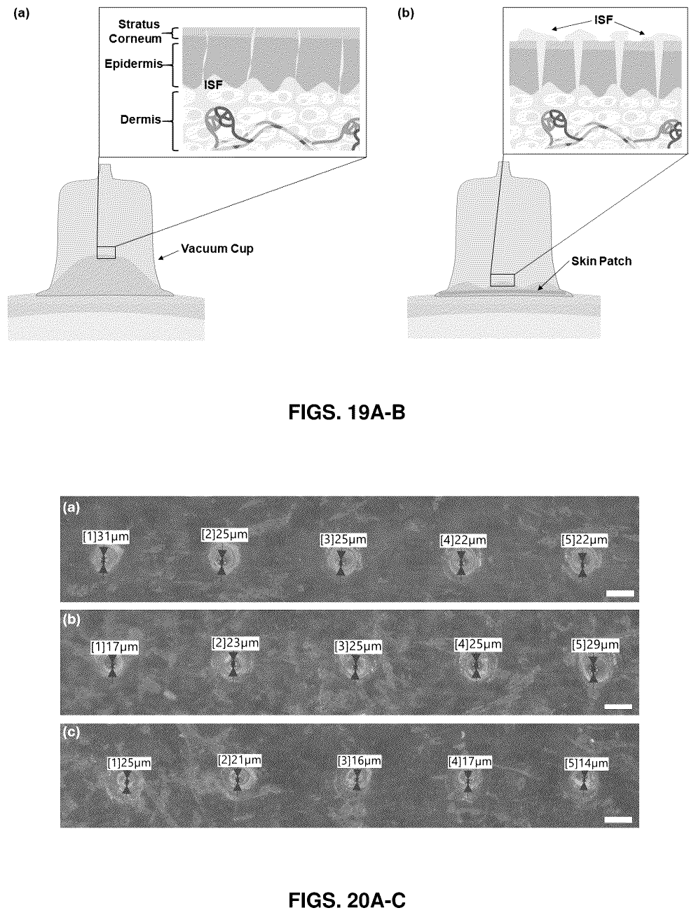

Proteomic analysis of dermal ISF. Dermal ISF and blood were sampled from five volunteers (demographics listed in Table S2) and analyzed for protein composition using LC-MS/MS. The inventors initially analyzed both fluids and found that they both contained a high level of abundant proteins, such as albumin and immunoglobulins. Therefore, the abundant proteins were removed from the fluids using a commercial protein depletion kit and re-analyzed. This analysis resulted in the identification of 2,195 distinct proteins, where 91.6% were common between both fluids, 4.7% were unique to blood serum, and 3.7% were unique to ISF (FIG. 9a). To determine the differential abundance of proteins in each fluid, protein identifications were filtered for statistical significance and relative fold-change, as shown in the volcano plot (FIG. 9b). The proteomic results were further analyzed using two online biomarker databases (OncoMX (Dingerdissen et al., 2020) and BIONDA (Turewicz et al., 2021)) to identify medically relevant biomarkers. From these databases, 610 proteins detected in both ISF and serum with similar abundance ratios (within a log₂ value of 0.50-2.0) are associated with diseases (Table S3). Of these proteins identified, 98 are classified in the NCI Early Detection Research Network (EDRN) biomarker database and 5 are approved biomarkers by the U.S. Food and Drug Administration (FDA), with 3 of these being both EDRN and FDA biomarkers. The abundance levels of proteins in ISF and serum determined by LC-MS/MS were based on relative measurements. Therefore, the absolute protein concentrations in the five paired ISF and serum samples were measured using a Bradford protein assay. From this analysis, the absolute concentration of proteins in ISF and serum were 46.50±8.25 mg mL⁻¹ and 74.90±6.23 mg mL⁻¹, respectively (FIGS. 19A-B), which is consistent with prior studies analyzing the total protein content in these fluids (Svedman et al., 2002; Reed & Rubin, 2010; Leeman et al., 2018; Sloop et al., 1987).

TABLE S2
Demographics of the 5 participants enrolled in the ISF collection
study whose samples were analyzed using LC-MS/MS.
Age (years)

| | |
|---|---|
| Mean ± SD | 29.6 ± 5.1 |
| Median | 28 |

Sex

| | |
|---|---|
| Male | 2 |
| Female | 3 |

Ethnicity

| | |
|---|---|
| Caucasian | 1 |
| Asian | 3 |
| Prefer not to answer | 1 |

TABLE S3
Proteins detected in both dermal ISF and blood with similar abundance
ratios (0.5-2.0) recognized as medically relevant biomarkers.

| | | Abundance | |
|---|---|---|---|
| | | Ratio | Category (if |
| Protein Name | Gene Symbol | (ISF:Blood) | applicable) |
| Acidic leucine-rich nuclear phosphoprotein 32 | ANP32B | 0.501 | |
| family member B | | | |
| Ribose-5-phosphate isomerase | RPIA | 0.507 | |
| Alpha-mannosidase 2 | MAN2A1 | 0.508 | |
| Large ribosomal subunit protein uL5 | RPL11 | 0.508 | |
| Probable ubiquitin carboxyl-terminal hydrolase FAF- | USP9X | 0.508 | |
| X | | | |
| Collectin-11 | COLEC11 | 0.509 | |
| Heat shock protein 105 kDa | HSPH1 | 0.509 | |
| Flotillin-2 | FLOT2 | 0.51 | |
| Eukaryotic peptide chain release factor GTP-binding | GSPT1 | 0.51 | |
| subunit ERF3A | | | |
| Qui oxidoreductase PIG3 | TP53I3 | 0.515 | |
| HLA class I histocompatibility antigen, C alpha | HLA-C | 0.52 | |
| chain | | | |
| 3-mercaptopyruvate sulfurtransferase | MPST | 0.523 | |
| Neural cell adhesion molecule 1 | NCAM1 | 0.523 | |
| Neurogenic locus notch homolog protein 2 | NOTCH2 | 0.525 | |
| Glucose-6-phosphate 1-dehydrogenase | G6PD | 0.527 | |
| Annexin A11 | ANXA11 | 0.529 | |
| Kinesin-like protein KIF14 | KIF14 | 0.53 | |
| Mannose-binding protein C | MBL2 | 0.53 | |
| E3 ubiquitin-protein ligase HUWE1 | HUWE1 | 0.533 | |
| Arginine--tRNA ligase, cytoplasmic | RARS1 | 0.534 | |
| Nucleosome assembly protein 1-like 4 | NAP1L4 | 0.536 | |
| 26S proteasome regulatory subunit 8 | PSMC5 | 0.537 | |
| Reticulon-4 | RTN4 | 0.539 | |
| Mannan-binding lectin serine protease 2 | MASP2 | 0.544 | |
| Galectin-3-binding protein | LGALS3BP | 0.545 | EDRN |
| Platelet-activating factor acetylhydrolase | PLA2G7 | 0.546 | |
| Insulin-like growth factor-binding protein 3 | IGFBP3 | 0.548 | EDRN |
| Eukaryotic translation initiation factor 2 subunit 1 | EIF2S1 | 0.549 | |
| Proteoglycan 4 | PRG4 | 0.55 | |
| Protein arginine N-methyltransferase 5 | PRMT5 | 0.552 | EDRN |
| Beta-Ala-His dipeptidase | CNDP1 | 0.553 | |
| Tsukushi | TSKU | 0.553 | |
| Anthrax toxin receptor 2 | ANTXR2 | 0.554 | |
| Keratin, type II cytoskeletal 7 | KRT7 | 0.554 | EDRN |
| Protein Z-dependent protease inhibitor | SERPINA10 | 0.555 | |
| Matrilin-2 | MATN2 | 0.557 | |
| Cholesteryl ester transfer protein | CETP | 0.56 | |
| 6-phosphogluconate | PGD | 0.56 | |
| dehydrogenase, decarboxylating | | | |
| Vitamin K-dependent protein S | PROS1 | 0.565 | EDRN |
| CD166 antigen | ALCAM | 0.567 | EDRN |
| BolA-like protein 2 | BOLA2; | 0.57 | |
| | BOLA2B | | |
| Mannan-binding lectin serine protease 1 | MASP1 | 0.57 | |
| Cation-independent mannose-6-phosphate receptor | IGF2R | 0.571 | |
| Immunoglobulin J chain | JCHAIN | 0.573 | |
| Tropomodulin-1 | TMOD1 | 0.576 | |
| Apolipoprotein E | APOE | 0.578 | |
| Lupus La protein | SSB | 0.578 | |
| Coiled-coil domain-containing protein 80 | CCDC80 | 0.579 | |
| Cathepsin S | CTSS | 0.579 | |
| C—C motif chemokine 18 | CCL18 | 0.582 | EDRN |
| Biliverdin reductase A | BLVRA | 0.584 | FDA |
| Nucleoside diphosphate kinase A | NME1 | 0.586 | |
| Pyridoxal phosphate homeostasis protein | PLPBP | 0.586 | |
| Low affinity immunoglobulin gamma Fc region | FCGR3B | 0.587 | |
| receptor III-B | | | |
| Hsp90 co-chaperone Cdc37 | CDC37 | 0.589 | |
| Complement C1q subcomponent subunit B | C1QB | 0.594 | |
| Nicotinate phosphoribosyltransferase | NAPRT | 0.595 | |
| Vascular endothelial growth factor receptor 2 | KDR | 0.596 | EDRN |
| T-complex protein 1 subunit eta | CCT7 | 0.597 | |
| Ubiquitin carboxyl-terminal hydrolase MINDY-3 | MINDY3 | 0.598 | |
| Importin subunit alpha-7 | KPNA6 | 0.599 | EDRN |
| GTP-binding nuclear protein Ran | RAN | 0.599 | |
| All-trans-retinol dehydrogenase [NAD(+)] ADH4 | ADH4 | 0.6 | |
| Apolipoprotein D | APOD | 0.6 | |
| Apolipoprotein F | APOF | 0.602 | |
| Band 4.1-like protein 3 | EPB41L3 | 0.602 | EDRN |
| C4b-binding protein alpha chain | C4BPA | 0.605 | |
| UDP-glucose 4-epimerase | GALE | 0.605 | |
| Coagulation factor X | F10 | 0.606 | |
| Mitogen-activated protein kinase 3 | MAPK3 | 0.609 | |
| Cullin-4A | CUL4A | 0.61 | |
| Integrin-linked protein kinase | ILK | 0.611 | |
| Malignant T-cell-amplified sequence 1 | MCTS1 | 0.612 | |
| Vitamin K-dependent protein Z | PROZ | 0.612 | |
| Histone-lysine N-methyltransferase 2D | KMT2D | 0.613 | |
| Glycogen phosphorylase, liver form | PYGL | 0.613 | |
| Inter-alpha-trypsin inhibitor heavy chain H3 | ITIH3 | 0.615 | |
| Creatine kinase M-type | CKM | 0.616 | EDRN |
| Indian hedgehog protein | IHH | 0.616 | |
| T-complex protein 1 subunit beta | CCT2 | 0.618 | |
| BMP-2-inducible protein kinase | BMP2K | 0.619 | |
| Filamin-A | FLNA | 0.626 | EDRN |
| Neurogenic locus notch homolog protein 3 | NOTCH3 | 0.626 | |
| Isochorismatase domain-containing protein 1 | ISOC1 | 0.629 | |
| Adapter molecule crk | CRK | 0.631 | |
| Receptor-type tyrosine-protein phosphatase eta | PTPRJ | 0.631 | |
| Angiotensin-converting enzyme | ACE | 0.633 | |
| T-complex protein 1 subunit zeta | CCT6A | 0.636 | |
| ATP-dependent 6-phosphofructokinase, platelet type | PFKP | 0.638 | |
| Optineurin | OPTN | 0.639 | |
| Adenosine 5′-monophosphoramidase HINT1 | HINT1 | 0.643 | |
| Inter-alpha-trypsin inhibitor heavy chain H1 | ITIH1 | 0.644 | |
| Phosphatidylcholine-sterol acyltransferase | LCAT | 0.644 | |
| Ras-related protein Rab-5A | RAB5A | 0.646 | EDRN |
| Thioredoxin reductase 1, cytoplasmic | TXNRD1 | 0.646 | |
| Integrin alpha-7 | ITGA7 | 0.647 | |
| Serine/threonine-protein kinase PAK 2 | PAK2 | 0.647 | |
| Contactin-4 | CNTN4 | 0.65 | |
| Large ribosomal subunit protein uL3 | RPL3 | 0.65 | |
| Stabilin-1 | STAB1 | 0.653 | |
| Pyruvate kinase PKLR | PKLR | 0.654 | |
| Proteasome activator complex subunit 2 | PSME2 | 0.654 | |
| Protein S100-A8 | S100A8 | 0.654 | |
| Ras-related protein Rap-1A | RAP1A | 0.656 | |
| Serum amyloid A-4 protein | SAA4 | 0.656 | |
| Sialic acid synthase | NANS | 0.658 | |
| Glutathione S-transferase Mu 2 | GSTM2 | 0.661 | EDRN |
| Latexin | LXN | 0.661 | |
| Matrix-remodeling-associated protein 5 | MXRA5 | 0.662 | EDRN |
| Glutaredoxin-1 | GLRX | 0.664 | EDRN |
| Protein HEG homolog 1 | HEG1 | 0.676 | |
| CD5 antigen-like | CD5L | 0.679 | |
| Clusterin | CLU | 0.68 | |
| Transferrin receptor protein 1 | TFRC | 0.68 | |
| Heme-binding protein 1 | HEBP1 | 0.682 | |
| Carboxypeptidase N subunit 2 | CPN2 | 0.683 | |
| Lymphocyte function-associated antigen 3 | CD58 | 0.684 | |
| Ubiquitin-like modifier-activating enzyme 6 | UBA6 | 0.685 | |
| Ubiquitin carboxyl-terminal hydrolase 15 | USP15 | 0.685 | |
| NBAS subunit of NRZ tethering complex | NBAS | 0.693 | |
| T-complex protein 1 subunit delta | CCT4 | 0.697 | |
| Cytoskeleton-associated protein 5 | CKAP5 | 0.697 | |
| COP9 signalosome complex subunit 2 | COPS2 | 0.7 | |
| Phosphoribosylformylglycinamidine synthase | PFAS | 0.7 | EDRN |
| Eukaryotic translation initiation factor 3 subunit E | EIF3E | 0.709 | EDRN |
| Apolipoprotein C-I | APOC1 | 0.711 | |
| Histone-arginine methyltransferase CARM1 | CARM1 | 0.714 | |
| Apolipoprotein B-100 | APOB | 0.717 | EDRN |
| Protein IMPACT | IMPACT | 0.718 | |
| Stathmin | STMN1 | 0.718 | |
| Threonine--tRNA ligase 1, cytoplasmic | TARS1 | 0.718 | |
| Ficolin-3 | FCN3 | 0.721 | |
| Apolipoprotein M | APOM | 0.727 | |
| Complement C1r subcomponent-like protein | C1RL | 0.73 | |
| Alpha-mannosidase 2x | MAN2A2 | 0.733 | |
| Transportin-1 | TNPO1 | 0.734 | |
| DCN1-like protein 1 | DCUN1D1 | 0.736 | |
| Diphosphomevalonate decarboxylase | MVD | 0.736 | |
| Chronophin | PDXP | 0.736 | |
| Apolipoprotein A-I | APOA1 | 0.737 | EDRN |
| Myosin-10 | MYH10 | 0.738 | |
| Aldo-keto reductase family 1 member C3 | AKR1C3 | 0.739 | |
| Golgi-associated plant pathogenesis-related protein | [symbol] | [symbol] | |
| 1 | | | |
| Adenosylhomocysteinase | AHCY | 0.741 | |
| Complement C1s subcomponent | C1S | 0.744 | EDRN |
| N-acetyllactosaminide beta-1,3-N- | B3GNT2 | 0.745 | |
| acetylglucosaminyltransferase 2 | | | |
| Dopamine beta-hydroxylase | DBH | 0.745 | |
| Serine/threonine-protein phosphatase 2A activator | PTPA | 0.749 | EDRN |
| Desmoglein-2 | DSG2 | 0.751 | EDRN |
| Apoptosis regulator BAX | BAX | 0.752 | |
| Calpastatin | CAST | 0.752 | |
| GDH/6PGL endoplasmic bifunctional protein | H6PD | 0.754 | |
| Peptidoglycan recognition protein 1 | PGLYRP1 | 0.757 | |
| Ubiquitin carboxyl-terminal hydrolase 5 | USP5 | 0.757 | |
| Membrane primary amine oxidase | AOC3 | 0.76 | |
| Inter-alpha-trypsin inhibitor heavy chain H4 | ITIH4 | 0.761 | EDRN |
| Immunoglobulin lambda variable 3-21 | IGLV3-21 | 0.766 | |
| Adhesion G protein-coupled receptor F5 | ADGRF5 | 0.771 | |
| Vitronectin | VTN | 0.774 | |
| A disintegrin and metalloproteinase with | ADAMTS13 | 0.778 | |
| thrombospondin motifs 13 | | | |
| Carboxypeptidase N catalytic chain | CPN1 | 0.778 | |
| Low affinity immunoglobulin gamma Fc region | FCGR3A | 0.779 | |
| receptor III-A | | | |
| Beta-actin-like protein 2 | ACTBL2 | 0.782 | |
| Dipeptidyl peptidase 4 | DPP4 | 0.782 | EDRN |
| Intercellular adhesion molecule 1 | ICAM1 | 0.783 | EDRN |
| Serum paraoxonase/arylesterase 1 | PON1 | 0.783 | |
| Tyrosine-protein kinase receptor Tie-1 | TIE1 | 0.784 | |
| COP9 signalosome complex subunit 3 | COPS3 | 0.785 | |
| T-complex protein 1 subunit theta | CCT8 | 0.786 | |
| Insulin-like growth factor-binding protein complex | [symbol] | [symbol] | |
| acid labile subunit | | | |
| Nucleosome assembly protein 1-like 1 | NAP1L1 | 0.789 | |
| L-selectin | SELL | 0.797 | EDRN |
| Ankyrin-3 | ANK3 | 0.799 | |
| Intercellular adhesion molecule 2 | ICAM2 | 0.8 | |
| Keratin, type II cytoskeletal 80 | KRT80 | 0.802 | |
| Tubulin--tyrosine ligase-like protein 12 | TTLL12 | 0.804 | |
| Macrophage receptor MARCO | MARCO | 0.806 | |
| Fibronectin | FN1 | 0.807 | |
| Cadherin-5 | CDH5 | 0.812 | |
| Cullin-3 | CUL3 | 0.823 | |
| Neural cell adhesion molecule L1-like protein | CHL1 | 0.827 | |
| Complement C3 | C3 | 0.829 | EDRN |
| Alpha-2-macroglobulin | A2M | 0.831 | |
| Cholinesterase | BCHE | 0.831 | EDRN |
| Complement factor H-related protein 2 | CFHR2 | 0.831 | |
| Transitional endoplasmic reticulum ATPase | VCP | 0.832 | |
| Coagulation factor XI | F11 | 0.833 | |
| Vascular cell adhesion protein 1 | VCAM1 | 0.834 | EDRN |
| Argininosuccinate lyase | ASL | 0.84 | |
| E3 ubiquitin-protein ligase RNF123 | RNF123 | 0.84 | |
| Plexin domain-containing protein 1 | PLXDC1 | 0.842 | |
| Importin subunit alpha-3 | KPNA4 | 0.844 | |
| ATP-dependent 6-phosphofructokinase, muscle type | PFKM | 0.844 | |
| Heterogeneous nuclear ribonucleoprotein D0 | HNRNPD | 0.848 | EDRN |
| Poly(rC)-binding protein 3 | PCBP3 | 0.848 | |
| Regulator of G-protein signaling 10 | RGS10 | 0.851 | |
| Angiopoietin-related protein 6 | ANGPTL6 | 0.853 | |
| Complement C1q tumor necrosis factor-related | C1QTNF3 | 0.855 | |
| protein 3 | | | |
| N-acetylgalactosaminyltransferase 7 | GALNT7 | 0.857 | EDRN |
| Intercellular adhesion molecule 3 | ICAM3 | 0.859 | |
| Elongin-B | ELOB | 0.86 | |
| Heparin cofactor 2 | SERPIND1 | 0.86 | |
| Annexin A3 | ANXA3 | 0.863 | EDRN |
| Complement C4-B | C4B | 0.863 | |
| Complement C4-B | C4B_2 | 0.864 | |
| Ras-related protein Rab-1B | RAB1B | 0.864 | |
| Eukaryotic translation initiation factor 2 subunit 3 | EIF2S3 | 0.866 | |
| Dual specificity protein phosphatase 14 | DUSP14 | 0.87 | |
| Tyrosine-protein kinase receptor UFO | AXL | 0.875 | |
| Platelet endothelial cell adhesion molecule | PECAM1 | 0.881 | |
| Dystonin | DST | 0.882 | |
| Receptor-type tyrosine-protein phosphatase F | PTPRF | 0.884 | EDRN |
| Importin subunit beta-1 | KPNB1 | 0.885 | EDRN |
| Immunoglobulin heavy variable 4-34 | IGHV4-34 | 0.888 | |
| Serum paraoxonase/lactonase 3 | PON3 | 0.891 | |
| Peptidyl-prolyl cis-trans isomerase NIMA- | PIN4 | 0.893 | |
| interacting 4 | | | |
| Ubiquitin carboxyl-terminal hydrolase 24 | USP24 | 0.895 | |
| 26S proteasome non-ATPase regulatory subunit 14 | PSMD14 | 0.899 | |
| Lysosome-associated membrane glycoprotein 2 | LAMP2 | 0.9 | |
| Transcobalamin-2 | TCN2 | 0.9 | |
| Ubiquitin-conjugating enzyme E2 N | UBE2N | 0.903 | |
| Histone H2A type 1-C | H2AC6 | 0.904 | |
| Integrin beta-1 | ITGB1 | 0.913 | EDRN |
| Soluble scavenger receptor cysteine-rich | SSC5D | 0.914 | |
| domain- containing protein SSC5D | | | |
| Insulin-like growth factor I | IGF1 | 0.915 | EDRN |
| Synaptobrevin homolog YKT6 | YKT6 | 0.915 | |
| Serine/threonine-protein phosphatase 2A 65 kDa | PPP2R1A | 0.917 | |
| regulatory subunit A alpha isoform | | | |
| Cadherin-6 | CDH6 | 0.918 | |
| Plasma protease C1 inhibitor | SERPING1 | 0.92 | |
| Cartilage intermediate layer protein 2 | CILP2 | 0.923 | |
| T-complex protein 1 subunit alpha | TCP1 | 0.923 | |
| Protein phosphatase 1A | PPM1A | 0.93 | |
| Afamin | AFM | 0.933 | |
| Fibrocystin-L | PKHD1L1 | 0.94 | |
| Syndecan-1 | SDC1 | 0.943 | EDRN |
| Voltage-dependent calcium channel subunit | CACNA2D1 | 0.946 | |
| alpha- 2/delta-1 | | | |
| Cartilage acidic protein 1 | CRTAC1 | 0.947 | EDRN |
| Protein AMBP | AMBP | 0.948 | EDRN |
| Beta-enolase | ENO3 | 0.954 | |
| ADP-ribosyl cyclase/cyclic ADP-ribose hydrolase 2 | BST1 | 0.956 | |
| Noelin | OLFM1 | 0.958 | EDRN |
| Properdin | CFP | 0.959 | |
| Glutamyl aminopeptidase | ENPEP | 0.959 | |
| Complement component C7 | C7 | 0.963 | EDRN |
| Prostaglandin E synthase 3 | PTGES3 | 0.966 | |
| Proteasome subunit alpha type-1 | PSMA1 | 0.967 | |
| Alpha-soluble NSF attachment protein | NAPA | 0.971 | |
| Neural cell adhesion molecule 2 | NCAM2 | 0.973 | |
| Immunoglobulin kappa variable 4-1 | IGKV4-1 | 0.975 | |
| Enolase-phosphatase E1 | ENOPH1 | 0.976 | |
| Immunoglobulin heavy constant alpha 1 | IGHA1 | 0.976 | |
| 26S proteasome regulatory subunit 4 | PSMC1 | 0.976 | |
| Ubiquitin-ribosomal protein eS31 fusion protein | RPS27A | 0.976 | EDRN |
| Ubiquitin-ribosomal protein eL40 fusion protein | UBA52 | 0.976 | EDRN |
| Polyubiquitin-B | UBB | 0.976 | EDRN |
| Polyubiquitin-C | UBC | 0.976 | EDRN |
| Phospholipid transfer protein | PLTP | 0.977 | EDRN |
| Complement factor H | CFH | 0.98 | |
| Chromodomain-helicase-DNA-binding protein 6 | CHD6 | 0.98 | |
| Tolloid-like protein 1 | TLL1 | 0.981 | |
| Receptor-type tyrosine-protein phosphatase gamma | PTPRG | 0.982 | |
| Aspartate aminotransferase, cytoplasmic | GOT1 | 0.986 | |
| Cadherin-13 | CDH13 | 0.987 | EDRN |
| Poliovirus receptor | PVR | 0.987 | |
| Hepatocyte growth factor-like protein | MST1 | 0.991 | |
| Haptoglobin-related protein | HPR | 0.993 | |
| Protein S100-A9 | S100A9 | 0.995 | |
| Endoglin | ENG | 0.997 | EDRN |
| Receptor-type tyrosine-protein phosphatase mu | PTPRM | 0.997 | |
| Glutathione reductase, mitochondrial | GSR | 0.998 | |
| Glia maturation factor gamma | GMFG | 0.999 | |
| Ubiquitin carboxyl-terminal hydrolase isozyme L3 | UCHL3 | 0.999 | |
| Immunoglobulin-binding protein 1 | IGBP1 | 1 | |
| Ribose-phosphate pyrophosphokinase 1 | PRPS1 | 1.001 | |
| Cadherin-2 | CDH2 | 1.002 | |
| Ceruloplasmin | CP | 1.003 | |
| Plasminogen | PLG | 1.005 | |
| Basigin | BSG | 1.007 | |
| Biotinidase | BTD | 1.007 | |
| Proteasome activator complex subunit 1 | PSME1 | 1.007 | |
| Proteasome subunit beta type-5 | PSMB5 | 1.012 | |
| PITH domain-containing protein 1 | PITHD1 | 1.014 | |
| Exostosin-2 | EXT2 | 1.017 | |
| 26S proteasome regulatory subunit 10B | PSMC6 | 1.018 | |
| Syntaxin-binding protein 2 | STXBP2 | 1.018 | |
| AP-2 complex subunit alpha-1 | AP2A1 | 1.019 | |
| Transcobalamin-1 | TCN1 | 1.02 | |
| Plexin domain-containing protein 2 | PLXDC2 | 1.027 | |
| Histidine-rich glycoprotein | HRG | 1.029 | |
| Hemicentin-1 | HMCN1 | 1.03 | |
| Prolow-density lipoprotein receptor-related protein 1 | LRP1 | 1.03 | |
| Guanine nucleotide-binding protein G(I)/G(S)/G(T) | GNB1 | 1.031 | |
| subunit beta-1 | | | |
| Ubiquitin-conjugating enzyme E2 variant 2 | UBE2V2 | 1.031 | |
| Integrin alpha-2 | ITGA2 | 1.036 | |
| Ubiquitin carboxyl-terminal hydrolase 11 | USP11 | 1.043 | |
| Procathepsin L | CTSL | 1.045 | |
| Rap guanine nucleotide exchange factor 2 | RAPGEF2 | 1.05 | |
| Cullin-1 | CUL1 | 1.054 | |
| Complement C1q subcomponent subunit A | C1QA | 1.056 | |
| Neuropilin-2 | NRP2 | 1.056 | |
| 26S proteasome non-ATPase regulatory subunit 3 | PSMD3 | 1.056 | |
| RuvB-like 1 | RUVBL1 | 1.058 | |
| Protein argonaute-2 | AGO2 | 1.062 | |
| Cysteine and histidine-rich domain-containing | CHORDC1 | 1.063 | |
| protein 1 | | | |
| Dynein axonemal heavy chain 17 | DNAH17 | 1.065 | |
| Histone H1.2 | H1-2 | 1.065 | |
| Histone H1.3 | H1-3 | 1.065 | |
| Mannosyl-oligosaccharide 1,2-alpha-mannosidase | [symbol] | [symbol] | |
| IA | | | |
| Triokinase/FMN cyclase | TKFC | 1.068 | |
| N-acetylglucosamine-1-phosphotransferase subunit | [symbol] | [symbol] | |
| gamma | | | |
| ATPase GET3 | GET3 | 1.072 | |
| Contactin-3 | CNTN3 | 1.073 | |
| ADP-ribosylhydrolase ARH3 | ADPRS | 1.074 | |
| Protein argonaute-3 | AGO3 | 1.074 | |
| Calpain-2 catalytic subunit | CAPN2 | 1.075 | |
| Immunoglobulin heavy constant gamma 1 | IGHG1 | 1.075 | |
| Alpha-1B-glycoprotein | A1BG | 1.076 | |
| Hyaluronan-binding protein 2 | HABP2 | 1.076 | |
| Fructose-bisphosphate aldolase B | ALDOB | 1.077 | |
| C-reactive protein | CRP | 1.078 | EDRN |
| Integrin beta-2 | ITGB2 | 1.078 | |
| Glucosamine-6-phosphate isomerase 2 | GNPDA2 | 1.079 | |
| E3 ubiquitin-protein ligase KCMF1 | KCMF1 | 1.081 | |
| Laminin subunit beta-1 | LAMB1 | 1.081 | |
| Plasma kallikrein | KLKB1 | 1.083 | |
| Alpha-1-antitrypsin | SERPINA1 | 1.087 | EDRN |
| Titin | TTN | 1.088 | |
| N-acetylmuramoyl-L-alanine amidase | PGLYRP2 | 1.097 | |
| Methylosome protein WDR77 | WDR77 | 1.097 | |
| Complement component C8 alpha chain | C8A | 1.099 | |
| Macrophage mannose receptor 1 | MRC1 | 1.101 | EDRN |
| Histone H2B type 1-J | H2BC11 | 1.102 | |
| Histone H2B type 2-E | H2BC21 | 1.102 | |
| Histone H2B type 1-B | H2BC3 | 1.102 | |
| 26S proteasome non-ATPase regulatory subunit 9 | PSMD9 | 1.102 | |
| Calreticulin | CALR | 1.105 | EDRN |
| Thyroxine-binding globulin | SERPINA7 | 1.109 | |
| Hepatocyte growth factor activator | HGFAC | 1.122 | |
| Farnesyl pyrophosphate synthase | FDPS | 1.124 | |
| Endoplasmin | HSP90B1 | 1.126 | EDRN |
| Monocyte differentiation antigen CD14 | CD14 | 1.128 | EDRN |
| Tropomyosin beta chain | TPM2 | 1.131 | |
| Contactin-1 | CNTN1 | 1.133 | |
| Alpha-1-antichymotrypsin | SERPINA3 | 1.135 | |
| Beta/gamma crystallin domain-containing protein 1 | CRYBG1 | 1.136 | EDRN |
| Ras-related protein Rab-11A | RAB11A | 1.136 | |
| Ras-related protein Rab-11B | RAB11B | 1.136 | |
| 6-phosphogluconolactonase | PGLS | 1.14 | |
| Multimerin-2 | MMRN2 | 1.142 | EDRN |
| Ciliary neurotrophic factor receptor subunit alpha | CNTFR | 1.148 | |
| Plasma serine protease inhibitor | SERPINA5 | 1.152 | |
| Sex hormone-binding globulin | SHBG | 1.152 | |
| Y-box-binding protein 2 | YBX2 | 1.153 | |
| Alpha-1-acid glycoprotein 2 | ORM2 | 1.154 | |
| Nuclear transport factor 2 | NUTF2 | 1.159 | |
| Glia maturation factor beta | GMFB | 1.16 | |
| Selenoprotein P | SELENOP | 1.169 | |
| Prolyl endopeptidase FAP | FAP | 1.174 | |
| Dynamin-3 | DNM3 | 1.177 | |
| Complement factor I | CFI | 1.18 | |
| Putative Polycomb group protein ASXL2 | ASXL2 | 1.189 | |
| Angiotensinogen | AGT | 1.19 | |
| Thymidylate kinase | DTYMK | 1.191 | |
| Low affinity immunoglobulin gamma Fc region | FCGR2C | 1.191 | |
| receptor II-c | | | |
| Glutathione peroxidase 3 | GPX3 | 1.191 | EDRN |
| 1,4-alpha-glucan-branching enzyme | GBE1 | 1.196 | |
| Radixin | RDX | 1.196 | |
| Annexin A7 | ANXA7 | 1.2 | |
| Kallistatin | SERPINA4 | 1.201 | |
| Exportin-2 | CSE1L | 1.202 | |
| Kininogen-1 | KNG1 | 1.208 | EDRN |
| Phosphatidylinositol 3,4,5-trisphosphate 5- | INPP5D | 1.209 | |
| phosphatase 1 | | | |
| SEC14-like protein 2 | SEC14L2 | 1.209 | |
| Eukaryotic translation initiation factor 3 subunit G | EIF3G | 1.214 | |
| Ras-related protein Rab-14 | RAB14 | 1.216 | |
| Serine/threonine-protein phosphatase 2A catalytic | PPP2CA | 1.217 | |
| subunit alpha isoform | | | |
| Glutathione S-transferase omega-1 | GSTO1 | 1.222 | |
| Complement factor B | CFB | 1.226 | EDRN |
| Lipopolysaccharide-binding protein | LBP | 1.226 | EDRN |
| Immunoglobulin heavy constant gamma 3 | IGHG3 | 1.234 | |
| NEDD8-activating enzyme E1 regulatory subunit | NAE1 | 1.239 | |
| N-acetylneuraminate lyase | NPL | 1.24 | |
| Casein kinase II subunit alpha | CSNK2A1 | 1.242 | EDRN |
| Fibrillin-1 | FBN1 | 1.243 | |
| Deleted in malignant brain tumors 1 protein | DMBT1 | 1.245 | |
| Corticosteroid-binding globulin | SERPINA6 | 1.249 | EDRN |
| Platelet-activating factor acetylhydrolase IB subunit | PAFAH1B2 | 1.255 | |
| alpha2 | | | |
| Microtubule-actin cross-linking factor 1, isoforms | MACF1 | 1.257 | |
| 1/2/3/4/5 | | | |
| C-1-tetrahydrofolate synthase, cytoplasmic | MTHFD1 | 1.26 | |
| Endoplasmic reticulum chaperone BiP | HSPA5 | 1.261 | |
| ADP/ATP translocase 1 | SLC25A4 | 1.262 | |
| ADP/ATP translocase 3 | SLC25A6 | 1.262 | |
| EGF-containing fibulin-like extracellular matrix | EFEMP2 | 1.264 | |
| protein 2 | | | |
| Retinoic acid receptor responder protein 2 | RARRES2 | 1.268 | |
| Histone H2B type 1-K | H2BC12 | 1.269 | |
| Histone H2B type F-S | H2BC12L | 1.269 | |
| Histone H2B type 1-L | H2BC13 | 1.269 | |
| Histone H2B type 1-N | H2BC15 | 1.269 | |
| Histone H2B type 1-D | H2BC5 | 1.269 | |
| Histone H2B type 1-H | H2BC9 | 1.269 | |
| Bone marrow proteoglycan | PRG2 | 1.269 | |
| Aminopeptidase N | ANPEP | 1.273 | |
| Neural cell adhesion molecule L1 | L1CAM | 1.274 | EDRN |
| Eukaryotic translation initiation factor 4E | EIF4E | 1.275 | |
| Interleukin-6 receptor subunit alpha | IL6R | 1.28 | EDRN |
| Adenosine deaminase 2 | ADA2 | 1.282 | |
| Hepatocyte growth factor receptor | MET | 1.282 | BOTH |
| Endoplasmic reticulum aminopeptidase 2 | ERAP2 | 1.285 | |
| Pantothenate kinase 2, mitochondrial | PANK2 | 1.287 | |
| Interleukin-1 receptor accessory protein | IL1RAP | 1.29 | |
| CCHC-type zinc finger nucleic acid binding protein | CNBP | 1.301 | |
| Alpha-2-antiplasmin | SERPINF2 | 1.31 | |
| Fumarylacetoacetase | FAH | 1.314 | |
| Early endosome antigen 1 | EEA1 | 1.315 | |
| Leukocyte elastase inhibitor | SERPINB1 | 1.315 | |
| Fetuin-B | FETUB | 1.318 | |
| Oxysterol-binding protein 1 | OSBP | 1.324 | |
| Bifunctional purine biosynthesis protein ATIC | ATIC | 1.328 | |
| Transthyretin | TTR | 1.328 | EDRN |
| Hypoxia up-regulated protein 1 | HYOU1 | 1.332 | |
| Periplakin | PPL | 1.333 | |
| Bifunctional phosphoribosylaminoimidazole | PAICS | 1.334 | |
| carboxylase/phosphoribosylaminoimidazole | | | |
| succinocarboxamide synthetase | | | |
| Plexin-D1 | PLXND1 | 1.344 | |
| V-type proton ATPase subunit B, kidney isoform | ATP6V1B1 | 1.347 | |
| Complement C4-A | C4A | 1.352 | |
| Macrophage colony-stimulating factor 1 receptor | CSF1R | 1.352 | |
| Coagulation factor XII | F12 | 1.352 | |
| ATP-dependent RNA helicase A | DHX9 | 1.361 | |
| AP-2 complex subunit alpha-2 | AP2A2 | 1.362 | |
| Desmocollin-2 | DSC2 | 1.362 | EDRN |
| Kell blood group glycoprotein | KEL | 1.375 | |
| Microtubule-associated protein RP/EB family | MAPRE2 | 1.376 | |
| member 2 | | | |
| Mast/stem cell growth factor receptor Kit | KIT | 1.382 | BOTH |
| Heterogeneous nuclear ribonucleoprotein Q | SYNCRIP | 1.383 | |
| Tenascin-X | TNXB | 1.386 | |
| Parkinson disease protein 7 | PARK7 | 1.393 | EDRN |
| Collagen alpha-2(V) chain | COL5A2 | 1.397 | |
| Aflatoxin B1 aldehyde reductase member 2 | AKR7A2 | 1.4 | |
| Carboxypeptidase B2 | CPB2 | 1.404 | |
| NEDD8-conjugating enzyme Ubc12 | UBE2M | 1.405 | |
| Axin interactor, dorsalization-associated protein | AIDA | 1.406 | |
| Argininosuccinate synthase | ASS1 | 1.407 | |
| Complement component C9 | C9 | 1.408 | EDRN |
| Tropomyosin alpha-4 chain | TPM4 | 1.417 | |
| Fibroblast growth factor receptor 1 | FGFR1 | 1.419 | |
| Antithrombin-III | SERPINC1 | 1.42 | |
| Neutrophil gelatinase-associated lipocalin | LCN2 | 1.421 | EDRN |
| Complement receptor type 2 | CR2 | 1.426 | |
| Eukaryotic translation initiation factor 1A, X- | EIF1AX | 1.426 | |
| chromosomal | | | |
| Nardilysin | NRDC | 1.427 | |
| Peripherin | PRPH | 1.43 | EDRN |
| Sulfhydryl oxidase 1 | QSOX1 | 1.43 | EDRN |
| Ras-related protein Rab-8A | RAB8A | 1.432 | |
| EH domain-containing protein 2 | EHD2 | 1.435 | |
| Coronin-1A | CORO1A | 1.436 | |
| Tropomyosin alpha-1 chain | TPM1 | 1.443 | |
| Complement factor H-related protein 1 | CFHR1 | 1.447 | |
| Superoxide dismutase [Mn], mitochondrial | SOD2 | 1.452 | |
| Heat shock 70 kDa protein 4 | HSPA4 | 1.453 | |
| Pancreatic alpha-amylase | AMY2A | 1.457 | EDRN |
| DNA damage-binding protein 1 | DDB1 | 1.463 | |
| 4F2 cell-surface antigen heavy chain | SLC3A2 | 1.463 | |
| Interleukin-6 receptor subunit beta | IL6ST | 1.469 | |
| MAM domain-containing protein 2 | MAMDC2 | 1.481 | |
| ADAMTS-like protein 4 | ADAMTSL4 | 1.482 | |
| Epidermal growth factor receptor | EGFR | 1.482 | BOTH |
| Large ribosomal subunit protein uL13 | RPL13A | 1.483 | |
| Nuclear receptor-binding protein | NRBP1 | 1.486 | |
| Reticulon-3 | RTN3 | 1.488 | |
| Phospholysine phosphohistidine inorganic | LHPP | 1.492 | |
| pyrophosphate phosphatase | | | |
| Alpha-2-HS-glycoprotein | AHSG | 1.493 | |
| Copine-3 | CPNE3 | 1.493 | |
| Cell surface glycoprotein MUC18 | MCAM | 1.495 | EDRN |
| Alpha-actinin-3 | ACTN3 | 1.502 | |
| Angiogenin | ANG | 1.503 | EDRN |
| Histone H3.3 | H3-3A | 1.515 | |
| Histone H3.3 | H3-3B | 1.515 | |
| Beta-2-glycoprotein 1 | APOH | 1.516 | |
| ICOS ligand | ICOSLG | 1.518 | |
| Albumin | ALB | 1.52 | |
| Immunoglobulin heavy variable 3-23 | IGHV3-23 | 1.521 | |
| Receptor-type tyrosine-protein phosphatase zeta | PTPRZ1 | 1.521 | |
| Ubiquitin-conjugating enzyme E2 L3 | UBE2L3 | 1.526 | |
| Apolipoprotein A-IV | APOA4 | 1.527 | |
| Retinol-binding protein 4 | RBP4 | 1.527 | EDRN |
| ADP/ATP translocase 2 | SLC25A5 | 1.527 | |
| Interleukin-1 alpha | IL1A | 1.531 | |
| 2′,3′-cyclic-nucleotide 3′-phosphodiesterase | CNP | 1.532 | |
| Peptidyl-prolyl cis-trans isomerase | PIN1 | 1.533 | |
| NIMA- interacting 1 | | | |
| Small ribosomal subunit protein uS2 | RPSA | 1.533 | EDRN |
| Cadherin-10 | CDH10 | 1.534 | |
| Coactosin-like protein | COTL1 | 1.544 | |
| cAMP-dependent protein kinase catalytic subunit | PRKACA | 1.546 | |
| alpha | | | |
| Immunoglobulin kappa constant | IGKC | 1.55 | |
| Nesprin-2 | SYNE2 | 1.55 | |
| Cytoplasmic FMR1-interacting protein 1 | CYFIP1 | 1.551 | |
| CAD protein | CAD | 1.552 | |
| Zinc-alpha-2-glycoprotein | AZGP1 | 1.556 | EDRN |
| Adiponectin | ADIPOQ | 1.557 | EDRN |
| 2′-deoxynucleoside 5′-phosphate N-hydrolase 1 | DNPH1 | 1.558 | |
| Myosin-9 | MYH9 | 1.559 | |
| Ras-related protein Rab-2A | RAB2A | 1.559 | |
| Interleukin-1 receptor type 2 | IL1R2 | 1.561 | |
| Putative tenascin-XA | TNXA | 1.562 | |
| Hepatocyte growth factor-regulated tyrosine kinase | HGS | 1.563 | |
| substrate | | | |
| Serine/threonine-protein phosphatase 2A catalytic | PPP2CB | 1.564 | |
| subunit beta isoform | | | |
| Alpha-aminoadipic semialdehyde dehydrogenase | ALDH7A1 | 1.566 | |
| Dihydropyrimidinase-related protein 3 | DPYSL3 | 1.575 | EDRN |
| Leukocyte immunoglobulin-like receptor subfamily | [symbol] | [symbol] | |
| B member 2 | | | |
| Plasma membrane calcium-transporting ATPase 2 | ATP2B2 | 1.577 | |
| Plasma membrane calcium-transporting ATPase 3 | ATP2B3 | 1.577 | |
| Dihydropteridine reductase | QDPR | 1.577 | |
| CTP synthase 2 | CTPS2 | 1.582 | |
| Nucleoside diphosphate kinase B | NME2 | 1.587 | EDRN |
| cAMP-dependent protein kinase type II-alpha | PRKAR2A | 1.589 | |
| regulatory subunit | | | |
| Keratin, type I cytoskeletal 20 | KRT20 | 1.591 | |
| Alpha-1-acid glycoprotein 1 | HEL-S-153w | 1.593 | |
| Exportin-1 | XPO1 | 1.597 | |
| Thimet oligopeptidase | THOP1 | 1.604 | |
| Fibroblast growth factor-binding protein 2 | FGFBP2 | 1.606 | |
| Receptor-type tyrosine-protein phosphatase S | PTPRS | 1.613 | EDRN |
| Cartilage oligomeric matrix protein | COMP | 1.614 | |
| Endothelial protein C receptor | PROCR | 1.622 | |
| 26S proteasome non-ATPase regulatory subunit 4 | PSMD4 | 1.624 | |
| UTP--glucose-1-phosphate uridylyltransferase | UGP2 | 1.626 | |
| Pantetheinase | VNN1 | 1.627 | |
| Reversion-inducing cysteine-rich protein with | RECK | 1.637 | |
| Kazal motifs | | | |
| Large ribosomal subunit protein eL30 | RPL30 | 1.647 | |
| Serotransferrin | TF | 1.649 | EDRN |
| Insulin-like growth factor-binding protein 5 | IGFBP5 | 1.652 | FDA |
| Hemopexin | HPX | 1.655 | |
| Myocilin | MYOC | 1.66 | |
| Regenerating islet-derived protein 3-alpha | REG3A | 1.661 | EDRN |
| Serotransferrin | TF | 1.666 | EDRN |
| Exopolyphosphatase PRUNE1 | PRUNE1 | 1.667 | |
| Leukocyte immunoglobulin-like receptor subfamily | LILRA3 | 1.672 | |
| A member 3 | | | |
| Testin | TES | 1.678 | |
| Uromodulin | UMOD | 1.679 | |
| Anamorsin | CIAPIN1 | 1.686 | |
| Limbic system-associated membrane protein | LSAMP | 1.691 | |
| Ankyrin-2 | ANK2 | 1.696 | |
| ATP-dependent RNA helicase DDX1 | DDX1 | 1.698 | |
| Myosin regulatory light polypeptide 9 | MYL9 | 1.701 | |
| Platelet-derived growth factor receptor beta | PDGFRB | 1.702 | |
| Cysteine--tRNA ligase, cytoplasmic | CARS1 | 1.708 | |
| Serine protease 1 | PRSS1 | 1.709 | |
| S-methyl-5′-thioadenosine phosphorylase | MTAP | 1.71 | |
| Nicotinamide phosphoribosyltransferase | NAMPT | 1.711 | |
| Leucine-rich alpha-2-glycoprotein | LRG1 | 1.714 | EDRN |
| Xaa-Pro dipeptidase | PEPD | 1.717 | |
| Ribonuclease inhibitor | RNH1 | 1.719 | |
| Keratin, type II cytoskeletal 1 | KRT1 | 1.733 | |
| Mimecan | OGN | 1.734 | |
| Fibulin-1 | FBLN1 | 1.737 | EDRN |
| Calcium/calmodulin-dependent protein kinase type | [symbol] | [symbol] | |
| II subunit beta | | | |
| S-formylglutathione hydrolase | ESD | 1.745 | |
| Keratin, type II cytoskeletal 4 | KRT4 | 1.751 | |
| Small ribosomal subunit protein eS27 | RPS27 | 1.757 | |
| Probable ATP-dependent RNA helicase DDX6 | DDX6 | 1.764 | |
| Proteasome subunit alpha type-5 | PSMA5 | 1.775 | |
| Complement component C1q receptor | CD93 | 1.779 | |
| Proteasome subunit alpha type-7 | PSMA7 | 1.779 | |
| A-kinase anchor protein 9 | AKAP9 | 1.78 | |
| Proteasome subunit beta type-4 | PSMB4 | 1.787 | |
| Glycerol-3-phosphate phosphatase | PGP | 1.788 | |
| Protein kinase C-binding protein NELL2 | NELL2 | 1.79 | |
| Rho GDP-dissociation inhibitor 2 | ARHGDIB | 1.795 | |
| Thrombospondin-4 | THBS4 | 1.814 | |
| EH domain-containing protein 1 | EHD1 | 1.818 | |
| Serine/threonine-protein kinase PAK 1 | PAK1 | 1.821 | |
| Cyclin-dependent kinase 2 | CDK2 | 1.823 | |
| Nascent polypeptide-associated complex subunit | NACA | 1.826 | EDRN |
| alpha, muscle-specific form | | | |
| Leukocyte immunoglobulin-like receptor subfamily | LILRB5 | 1.831 | |
| B member 5 | | | |
| CD109 antigen | CD109 | 1.832 | |
| S-adenosylmethionine synthase isoform type-2 | MAT2A | 1.832 | |
| WD repeat-containing protein 1 | WDR1 | 1.833 | |
| Proteasome subunit alpha type-6 | PSMA6 | 1.838 | |
| Lactoylglutathione lyase | GLO1 | 1.84 | EDRN |
| Tyrosine-protein phosphatase non-receptor type 6 | PTPN6 | 1.847 | |
| Putative protein-lysine deacylase ABHD14B | ABHD14B | 1.848 | |
| Superoxide dismutase [Cu—Zn] | SOD1 | 1.851 | EDRN |
| Filamin-C | FLNC | 1.858 | EDRN |
| Sodium/potassium-transporting ATPase | ATP1A3 | 1.861 | |
| subunit alpha-3 | | | |
| Gamma-enolase | ENO2 | 1.864 | EDRN |
| Glutamine--tRNA ligase | QARS1 | 1.864 | |
| Adenine phosphoribosyltransferase | APRT | 1.866 | |
| Lysozyme C | LYZ | 1.866 | |
| Adenylyl cyclase-associated protein 1 | CAP1 | 1.872 | |
| Ribonuclease 4 | RNASE4 | 1.895 | |
| Plasma alpha-L-fucosidase | FUCA2 | 1.896 | |
| Kallikrein-10 | KLK10 | 1.897 | |
| Zymogen granule protein 16 homolog B | ZG16B | 1.898 | |
| Complement decay-accelerating factor | CD55 | 1.903 | |
| E3 ubiquitin-protein ligase TRIM58 | TRIM58 | 1.904 | |
| Endonuclease domain-containing 1 protein | ENDOD1 | 1.908 | |
| Alpha-N-acetylglucosaminidase | NAGLU | 1.908 | |
| Inositol monophosphatase 1 | IMPA1 | 1.931 | |
| Calpain-1 catalytic subunit | CAPN1 | 1.933 | EDRN |
| Tyrosine-protein phosphatase non-receptor type 11 | PTPN11 | 1.94 | EDRN |
| Lipopolysaccharide-responsive and beige-like | LRBA | 1.951 | |
| anchor protein | | | |
| Src substrate cortactin | CTTN | 1.953 | |
| Beta-1,4-galactosyltransferase 1 | B4GALT1 | 1.955 | |
| Heat shock cognate 71 kDa protein | HSPA8 | 1.958 | |
| Semaphorin-7A | SEMA7A | 1.958 | |
| Peptidyl-glycine alpha-amidating monooxygenase | PAM | 1.961 | |
| Protein disulfide-isomerase A3 | PDIA3 | 1.966 | |
| Immunoglobulin kappa variable 1-5 | IGKV1-5 | 1.968 | |
| Bifunctional coenzyme A synthase | COASY | 1.985 | |
| Protein FosB | FOSB | 1.993 | |

[symbol]

indicates data missing or illegible when filed

Detection of SARS-CoV-2 neutralizing antibodies in dermal ISF. The inventors analyzed dermal ISF samples from COVID-19 vaccinees for the presence of SARS-CoV-2 neutralizing antibodies using two commercial SARS-CoV-2 neutralization antibody tests. Two paired dermal ISF and blood samples were first tested using a LFIA test. Dark test and control lines were generated with both samples for each participant, indicating the presence of SARS-CoV-2 neutralizing antibodies (FIG. 9c). Dermal ISF from 15 COVID-19 vaccinees was also analyzed using an ELISA-based SARS-CoV-2 surrogate virus neutralization test kit to quantify the concentration of the SARS-CoV-2 neutralizing antibody in the samples. SARS-CoV-2 neutralization antibody was detected in dermal ISF from all vaccinees at concentrations ranging from 81-618 ng mL⁻¹ (FIG. 9d). These collective results provide evidence that dermal ISF is a source for biomarkers associated with vaccination status and further suggests that other molecular biomarkers associated infection and disease status are presented in ISF.

### Example 3—Discussion

Progress in the use of dermal ISF as a diagnostic fluid has been hampered by the lack of simple, rapid, and minimally invasive sampling methods capable of extracting larger quantities of fluid (Friedel et al., 2023a). A major limitation of existing MN-based ISF sampling techniques is that the collected fluid volumes are too low for biomolecular analysis using commercially available diagnostic immunoassays (e.g., ELISA, Western Blot, LFIA), which require at least 10-20 μL of fluid. Here, the inventors present the development of a rapid (25 min), simple and minimally invasive technique for sampling ample quantities of ISF from human skin, which was achieved by implementing several unique strategies. Existing MN-based ISF sampling methods employ MN arrays consisting of a few MNs, resulting in a small number of micropores generated in the skin, even with repeated MN insertion. In the inventors' approach, a high-density MN array was applied to the skin three times (MN insertions were not intentionally aligned), resulting in the generation of thousands of micropores from which ISF could be extracted. More importantly, the inventors hypothesize that the low sample volumes generated from the MN- and vacuum-assisted ISF sampling methods reported in prior studies is due to the high elasticity of human skin, which can deform excessively when vacuum pressure is applied (Kalra & Lowe, 2016), causing the micropores to close (FIG. 20a). Studies demonstrating the extraction of larger quantities (>6 μL) (Kim et al., 2021) of fluid through MN-generated micropores in excised animal skin or artificial skin models (Makvandi et al., 2021; Kim et al., 2018) utilized high vacuum pressures to induce the opening of the micropores. Applying such strong suction to human skin would be painful and cause skin injury. To overcome this challenge, the inventors developed a rigid patch that is adhered to the skin, which serves two important functions. First, the patch creates an air-tight seal between the skin and vacuum cup, enabling vacuum pressure to be maintained throughout the ISF extraction process. Second, the patch keeps the skin taut when suction is applied, which induces the opening of the micropores, enabling pressure-driven convection of ISF through the micropores (FIG. 20b). To validate the effectiveness of the skin patch for enhancing ISF extraction in human skin, the ISF sampling procedure was performed on volunteers without the patch. Upon applying suction to the vacuum cup, the skin deformed significantly (compared to when the patch was used) and no fluid was observed on the skin after 20 min. The mechanism for enhanced ISF extraction using the skin patch could be applied to other ISF sampling methods that have been reported in literature, which could lead to further improvements in their performance.

The inventors analyzed the amount of ISF extracted using different sized MNs and found that the 450 μm-long needles yielded the largest volume of dermal ISF compared with the 600 μm- and 750 μm-long needles. They attribute this to the 450 μm-long MNs creating larger diameter micropores compared to the 600 μm- and 750 μm-long needles, which allows for more ISF to flow through the micropores. A base diameter of 200 μm was used for all the MNs, therefore, longer MNs have a more slender profile than shorter MNs, thereby creating smaller micropores in the skin. This was confirmed by measuring the pore size generated by MNs with the three different lengths when penetrated into a wax-based membrane model (FIGS. 21A-I) (Larraneta et al., 2014).

Proteomic analysis of dermal ISF and blood collected from five volunteers resulted in the identification of 2,195 distinct proteins with the majority of these appearing in both fluids. Of those found, 610 proteins detected in both ISF and serum with similar abundance ratios are recognized as medically relevant biomarkers according to the BIONDA and OncoMX databases. These biomarkers include ones for various types of cancers, neurodevelopmental disorders, inflammatory diseases, genetic disorders, and more. These data indicate that dermal ISF may be a source for many of the same biomarkers associated with illness, infection and vaccination status that are present in blood. While the inventors observed a significant overlap in protein composition in both fluids, there was also a small (˜3.8%) subset of proteins that were only detected in dermal ISF, indicating that ISF could provide unique diagnostic and health information that cannot be obtained from blood. Among these are proteins associated with inflammation (i.e., interleukin-37), physiological responses such as electrolyte secretion (i.e., calcium activated chloride channel regulator 4), and cancers such as cutaneous T-cell lymphoma (i.e., melanoma inhibitory activity protein 2). To further showcase the utility of this sampling technique for diagnostic testing, dermal ISF was collected from COVID-19 vaccinees and analyzed for SARS-CoV-2 neutralizing antibodies using two commercially available immunoassays. Using the LFIA-based test, SARS-CoV-2 neutralizing antibodies could be detected in a rapid (˜15 min) and simple manner while SARS-CoV-2 antibody levels could be quantified in the ISF samples using the ELISA-based test.

The sampling technique reported in this work represents a notable improvement over existing MN-based ISF sampling methods in the ability to rapidly extract larger ISF volumes in a minimally invasive manner without the use of specialized equipment. A comparison of this technique with other MN-based techniques for sampling ISF from human skin is presented in Table S4. In addition to its enhanced effectiveness in sampling dermal ISF, this technique was well tolerated by all participants with only minor adverse effects that completely resolved within one day. Furthermore, participants rated the sampling technique as being nearly pain-free, potentially making it a more acceptable sampling method for diagnostic testing, particularly by individuals with needle and blood phobias. Additional studies to further optimize the MN parameters and sample collection procedure could enhance the reliability of ISF collection (e.g., reduce variability). Lastly, the inventors envision that this technique could be used to collect dermal ISF from individuals with various infections and medical conditions to identity ISF-based biomarkers, including proteins, nucleic acids and exosomes, associated with those diseases, which would advance progress in the use of dermal ISF for diagnostic testing.

TABLE S4
Comparison of MN-based techniques
for sampling ISF from human skin.

| | Equipment | ISF | | Average | |
|---|---|---|---|---|---|
| | Needed for ISF | Extraction | | Collection | |
| Type of MN | Extraction | Time | | Volume (μL) | Reference |
| Hydrogel | None | 6 | hr | — | 26 |
| Hollow | None | 5 | min | 1.1 | 16 |
| Hollow | None | 15-20 | min | <1 | 27 |
| Hollow | Capillary tubes | 1-2 | hr | 16 | 9.13 |
| Solid | Electrical | 20 | min | 2.3 ± 2.1 | 8 |
| | vacuum pump | | | | |
| Solid | Electrical | 20 | min | 3.4 ± 3.2 | 15 |
| | vacuum pump | | | | |
| Solid | Vacuum cup | 20 | min | 20.8 ± 19.4 | This work |
| | and hand pump | | | | |

### Example 4—Materials & Methods

Design and fabrication of the MN array. The MN array is comprised of solid, conical MNs made from polymerized SU-8 photoresist coated with 1.5 m of parylene for enhanced mechanical strength and biocompatibility (Chen & Lee, 2021; Kuppusami & Oskouei, 2015). The MNs were designed to have a compact profile to minimize the discomfort when inserted into skin. Each MN has a based diameter of 200 m and height of 450 μm. The MNs are configured in a two-dimensional 10×10 array to multiply the number of micropores generated per insertion, with a needle-to-needle spacing of 400 m. The overall size of the MN array is 7.5×7.5 mm.

The master MN array was 3-D printed on a Photonic Professional GT lithography system (NanoScribe, MA, USA). Replica MN arrays were fabricated via centrifugation-assisted replica molding where master molds were constructed from PDMS (Sylgard 184, Dow, MI, USA) mixed at a 1:10 (curing agent-to-elastomer) ratio. The PDMS was degassed for 30 min, poured over the master array, and heated in a convection oven at 80° C. for 2 hr. Cured PDMS was cut into individual molds using a razor blade and cleaned in 70% isopropanol. SU-8 2025 photoresist (Kayaku Advanced Materials, MA, USA) was poured into the PDMS molds and centrifuged at 4,000 g for 15 min to create replica MN arrays. Replicas were cured under a 50 W UV (365 nm) lamp for 3 min, then coated with 1.5 m of parylene using a Labcoater 2 parylene deposition system (Specialty Coating Systems, IN, USA). 3 mm thick PMMA (McMaster Carr, IL, USA) was attached to the backside of the MN arrays to enhance their rigidity. MNs were inspected and imaged using a VHX-7000 optical microscope (Keyence Corporation, Osaka, Japan).

Preparation of AuNP-anti-human IgG conjugates. 200 μL of 30 nm-diameter AuNPs in solution (OD-50; Millipore Sigma, MA, USA) was aliquoted into a low-bind microcentrifuge tube (Eppendorf, Hamburg, Germany). 1-ethyl-3-(3-dimethylaminopropyl)carbodiimide (EDC) (Thermo Fisher Scientific, MA, USA) and N-hydroxysuccinimide (NHS) (Thermo Fisher Scientific) were prepared at 10 mg/mL in deionized water. 40 μL of EDC and 80 μL of NHS were added to the AuNP solution, incubated on a shaker at room temperature for 30 min, and then centrifuged at 10,000 g for 10 min. The supernatant was removed, and the pellet was resuspended in 200 μL of reaction buffer to wash away excess EDC and NHS. The solution was vortexed and centrifuged at 10,000 g for 10 min. The supernatant was removed and 200 μL of fresh reaction buffer was added. 1.55 μL of goat anti-human IgG (1.3 mg/mL; Jackson ImmunoResearch, PA, USA) was added to the solution, followed by 3 hr of incubation on a shaker at room temperature. After incubation, 2 μL of quencher was added and incubated for 10 min, followed by 10 min of centrifugation at 10,000 g. The supernatant was removed, and the pellet was resuspended in 200 μL of reaction buffer. The AuNP-anti-human IgG conjugate concentration was adjusted to OD-20 by adding 500 μL of conjugate diluent to the AuNP solution. Prepared AuNP-anti-human IgG conjugate solution was stored at 20° C.

Preparation of the conjugate release pad. Glass fiber strips (Millipore Sigma, MA, USA) were soaked in a PBS solution containing 10% sucrose (Millipore Sigma) in PBS, 2% bovine serum albumin (BSA) (Millipore Sigma) in PBS, and 0.25% Tween-20 (Millipore Sigma) in deionized water for 1 hr at 20° C. The strips were dried at 37° C. for 2 hr and hand-cut into 3 mm wide pads. 3 μL of AuNP-anti-human IgG conjugate solution was dispensed onto the conjugate release pads, dried at 37° C. for 2 hr and stored at 20° C. with desiccant.

Preparation of the nitrocellulose membrane. Nitrocellulose membrane (GE Healthcare, IL, USA) was adhered to a 60 mm×300 mm backing card (DCN Dx, CA, USA). Solutions of tetanus toxoid antigen reconstituted in deionized water (3 mg/mL; Enzo Life Sciences, NY, USA) and rabbit anti-goat IgG (H/L) in PBS (1 mg/mL; Bio-Rad Antibodies, CA, USA) were dispensed onto the membrane to generate test and control lines, respectively, using an automated liquid dispensing platform (BioDot XYZ3060, CA, USA). The membrane was dried at 37° C. for 2 hr.

Assembly of the lateral flow test strip. A 3 mm×20 mm cellulose absorbent pad (Millipore Sigma) was adhered to the backing card slightly overlapping (˜1 mm) the end of the prepared nitrocellulose membrane. The card was cut into 3 mm wide strips using a guillotine cutter (BioDot, CA, USA). Prepared strips were stored in 20° C. with desiccant.

Fabrication and assembly of the skin patch. The skin patch was designed using AutoCAD (Autodesk, CA, USA) and Solidworks (SolidWorks Corp., MA, USA) software. The microfluidic substrate was fabricated from 3 mm thick PMMA (McMaster Carr) and microchannels were etched into the substrate using a CNC micro-milling machine (Minitech Machinery Corporation, GA, USA). A CO₂ laser cutter (Universal Laser System, Inc., AZ, USA) was used to create the vacuum and sampling ports in the microfluidic substrate, double-sided pressure-sensitive tape (3M, MN, USA), PET film (Optiazure) and bandage tape (3M). The LFIA test strip was inserted into the microfluidic substrate and a prepared conjugate release pad was placed at the front of the strip. The assembled test strip was enclosed within the patch using PET film and double-sided pressure-sensitive tape. The PMMA-LFIA-PET assembly was sandwiched between two layers of medical-grade tape, securing it within the patch.

MN penetration testing. Cadaver porcine skin with hair, fat and subcutaneous tissue removed was purchased from Animal Technologies, Inc. (TX, USA). The skin was cut into 10 cm×10 cm sections, vacuum sealed, and stored at −20° C. Prior to testing, a frozen skin section was thawed at room temperature and mounted onto foil-wrapped cardboard using safety pins. MNs tips were coated in blue ink and the MN array was inserted into the skin section using a MN applicator (Micropoint Technologies, Singapore). MN insertion wounds were visualized using a Keyence VHX-7000 microscope.

Histological analysis was performed on porcine skin sections following MN insertion. MNs were coated with Trypan blue (Sigma-Aldrich, MA, USA) in glycerol (Sigma-Aldrich) solution, and the MN array was inserted into the skin section using a MN applicator. The skin sample was fixed in a 10% formalin solution (Sigma-Aldrich) for at least 48 hr, transferred and stored in a 70% ethanol solution. The sample was then embedded in paraffin (Sigma-Aldrich), dehydrated, sectioned, and stained with hematoxylin and eosin (H&E). Optical images of H&E-stained skin sections were captured using a Keyence VHX-7000 microscope.

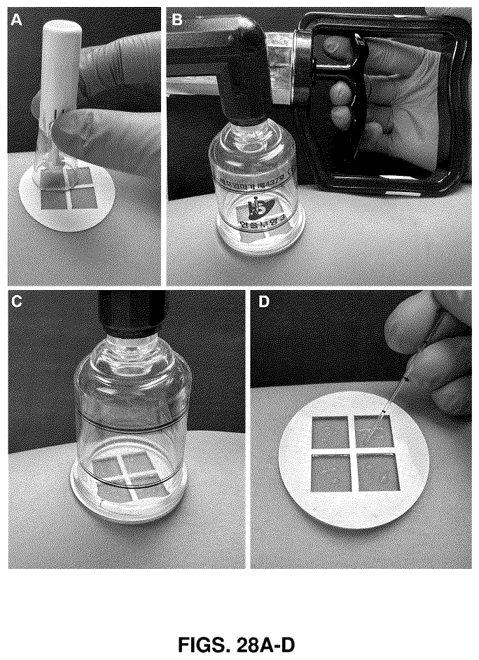

ISF and blood collection from human volunteers. Dermal ISF and fingerstick blood was collected from volunteers, which was performed under the guidance and approval from the Rice University Institutional Review Board (IRB-FY2021-147). Potential participants were provided with informed consent to participate in the study. Participants were explained the entirety of the sample collection process prior to beginning the study and informed consent was obtained from each individual. Criteria for participation was as follows: healthy adults or Rice University students ages 18 or older with no blood clotting disorders (including hemophilia, or factor II, V, VII, X, or XII deficiencies) or known skin allergies to medical adhesives. For ISF collection, the participant's forearm was cleaned using an alcohol prep pad (Fisher Healthcare, MA, USA). An adhesive stencil with cutouts for the MN insertion sites was adhered to the forearm and the MN array was applied two times at the insertion sites using a MN applicator (FIG. 28A). A rigid PMMA plate with cutouts at the MN insertion sites was attached to the stencil, followed by attachment of a vacuum cup (FIG. 28B). Vacuum pressure was generated inside the cup using a hand pump (Hansol Medical, South Korea) (FIG. 28C). After 20 min, the vacuum cup was removed and the extracted ISF was collected using capillary tubes (Thermo Fisher Scientific, MA, USA and Drummond Scientific Company, PA, USA) (FIG. 28D). The collected ISF was transferred to a low-bind microcentrifuge tube, incubated at room temperature for 1 hr and centrifuged at 10,000 g for 10 min. The supernatant was collected for analysis. Blood samples were obtained via fingerstick using a lancing device (Bayer Microlet) and 30 G lancets (CareTouch). Blood was collected in capillary tubes, transferred to a low-bind microcentrifuge tube, incubated for 1 h at room temperature, and centrifuged at 10,000 g for 10 min. The purified serum sample was transferred to a new low-bind microcentrifuge tube for analysis.

Anti-tetanus toxoid IgG quantification in blood and ISF samples. Anti-tetanus toxoid IgG levels were measured in dermal ISF and blood samples using a human anti-tetanus toxoid IgG ELISA kit (Alpha Diagnostics, TX, USA). Measurements were performed according to the manufacturer's instructions. Briefly, 1 μL of sample was combined with 100 μL of sample diluent and the mixture was dispensed into microwells pre-coated with tetanus toxoid antigen. The solution was gently mixed for 5 s, incubated at 37° C. for 60 min, and aspirated and blotted onto absorbent paper. The microwells were washed three times with 300 μL of diluted wash buffer. Next, 100 μL of anti-IgG HRP conjugate was added to each microwell, mixed for 5 s, and incubated at room temperature for 30 min. The aspiration and wash steps were repeated, then 100 L of TMB solution was added to each microwell. The solution was mixed for 5 s and incubated in the dark at room temperature for 15 min. 100 μL of stop solution was added to each microwell and gently mixed for 5 s, then the absorbance values were measured at 450 nm using a Biotek Epoch absorbance reader. A standard curve was calculated and used to determine the anti-tetanus toxoid IgG concentration in the samples.

Characterization of fluid flow through the skin patch. The inventors assessed the fluid flow characteristics through the skin patch using an artificial skin model (Makvandi et al., 2021). Briefly, 2% agar gel (Sigma-Aldrich) solution was boiled, poured into a 100-mm Petri dish, cured at room temperature, and stored at 4° C. Blue dye solution was dispensed on top of the agar gel and covered by Parafilm (Bemis Company, Inc., WI), which was carefully stretched over the Petri dish to prevent the entrapment of air bubbles. To initiate the experiment, an MN array was applied to the artificial skin using a MN applicator, followed by the attachment of the patch. A vacuum cup was attached to the vacuum port and vacuum pressure was generated using a hand pump. Video recordings and frame extractions were performed using an iPhone 14 Pro.

Evaluating the sensitivity and selectivity of the lateral flow immunoassay. For sensitivity testing, 15 μL of tetanus toxoid standard IgG (Alpha Diagnostics) at varying concentrations was dispensed onto the conjugate pad of the lateral flow test strip. For selectivity testing, measurements were performed by dispensing 15 μL of tetanus toxoid standard IgG (Alpha Diagnostics), diphtheria toxoid IgG (Virion, Wurzburg, Germany) or Bordetella pertussis toxin IgG (Virion, Wurzburg, Germany) at 0.1 IU/mL onto the conjugate pad. Images of the test results were obtained using a Canon CanoScan 9000F scanner.

Anti-tetanus toxoid IgG detection in dermal ISF using the skin patch. For this proof-of-concept measurement, the volunteer's anterior forearm was first cleaned using an alcohol prep pad and the MN array was applied into the skin using a MN applicator. Next, the patch was adhered to the skin and a vacuum cup was attached to the vacuum port of the patch. Vacuum pressure was generated using a hand pump. After 18 min, the vacuum cup was detached from the patch, the patch was peeled off and the forearm was cleaned using a fresh alcohol prep pad. Photographs of the volunteer's forearm were obtained at various time intervals after testing using an iPhone 14 Pro.

### Example 5—Results

Design of the skin patch. This device consists of a colloid gold-based LFIA integrated within a skin patch, which is comprised of a PMMA microfluidic network and PET film sandwiched between three layers of adhesive tape (FIG. 22A). The lateral flow test strip is based on a conventional LFIA architecture (Parolo et al., 2020), and consists of a glass fiber conjugate release pad, cellulose absorbent pad, and nitrocellulose membrane on a polyvinyl chloride backing card. The conjugate release pad contains AuNPs conjugated with anti-human IgG and the nitrocellulose membrane contains immobilized tetanus toxoid antigen and rabbit anti-goat IgG representing the test line and control line, respectively. During testing, anti-tetanus toxoid IgG in the sample binds to the AuNP-anti-human IgG conjugates on the conjugate pad forming AuNP-anti-human IgG-anti-tetanus toxoid IgG conjugates, which migrate toward the test line where they are captured to generate a red line. Uncaptured AuNP-anti-human IgG conjugates subsequently bind to the control line to generate a second red line, verifying the test result. The intensity of the test line is correlated with the concentration of anti-tetanus toxoid IgG in the sample where higher anti-tetanus toxoid IgG levels result in the generation of darker lines. Based on this detection scheme, this assay was designed to generate a test line (denoting a “positive” result) only when the anti-tetanus toxoid IgG concentration in the sample is equal or higher than the protective threshold concentration, indicating that the individual possesses sufficient protection against tetanus infection. When the anti-tetanus toxoid IgG concentration is below the protective threshold concentration, then no test line is generated (denoting a “negative” test result), which indicates that the individual possesses inadequate immunity against tetanus infection and requires a booster vaccine.

The microfluidic network is fabricated from 3 mm thick PMMA which contains cutouts for the fluidic channels, which are connected to three 6×6 mm sampling ports and a vacuum port (ø=18 mm). The LFIA test strip is secured within a 3 mm wide channel in the microfluidic network using the PET film and double-sided pressure-sensitive tape, and the PMMA-LFIA-PET assembly is sandwiched between two layers of medical-grade tape (the top layer is bandage tape, and the bottom layer is double-sided adhesive tape). The topside of the patch contains cutouts for the sampling ports (to facilitate alignment with the MN insertion sites), test (“T”) and control (“C”) line indicators, a result window, and the vacuum port (FIG. 22B). The internal view of the patch reveals the configuration of the sampling ports, microchannels, LFIA strip, and the vacuum port (FIG. 22C). The adhesive backing enables the patch to remain securely attached to the skin during testing.

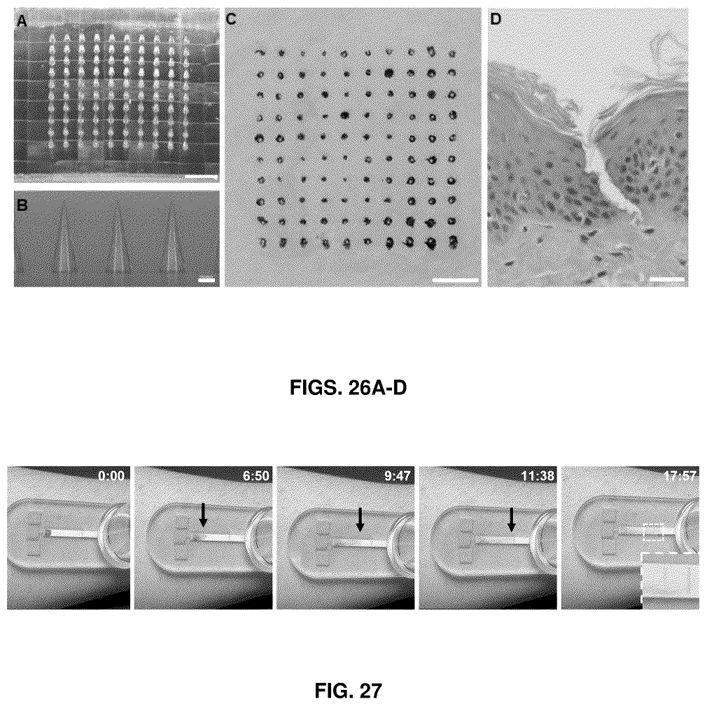

Characterization of MN penetration. The inventors briefly assessed the capability of the MN array to generate micropores in skin via insertion into cadaver porcine skin, which was used as an anatomically and biochemically similar model to human skin (Schmook et al., 2001). Prior to skin insertion, MNs were coated with blue ink for improved visualization. Distinct pores were generated by each MN, which were confined to the needle penetration sites with no impact to the surrounding tissue (FIG. 26C). Histological analysis was performed to evaluate the effects of MN penetration in skin tissue. Each MN insertion site was characterized by a conical micropore that pierced through the epidermis (FIG. 26D). The formation of these cavities provides access to ISF in the upper dermis, while avoiding the dense collection of nerves and vascular structures located in the lower dermis (Waghule et al., 2019).

Analysis of dermal ISF and blood for anti-tetanus toxoid IgG. Paired dermal ISF and blood samples from four healthy volunteers (demographics are listed in Table A) were analyzed for anti-tetanus toxoid IgG levels using a commercial ELISA kit. Antibodies to tetanus toxoid were detected in ISF of all the volunteers at concentrations from ˜0.6 to 1.1 IU/mL (FIG. 23). Anti-tetanus toxoid IgG levels in ISF were well-correlated with those in blood where the average ISF-to-blood ratio for all the samples was ˜0.8. These results provide compelling evidence that immune antibodies generated in response to infections and vaccinations are present in dermal ISF.

TABLE A
Demographics of volunteers whose blood and ISF were
collected and analyzed for anti-tetanus toxoid IgG.

| Participant ID | Age | Gender | Ethnicity |
|---|---|---|---|
| 1 | 25 | Female | White |
| 2 | 19 | Female | Asian |
| 3 | 23 | Male | White |
| 4 | 38 | Male | Prefer not to answer |

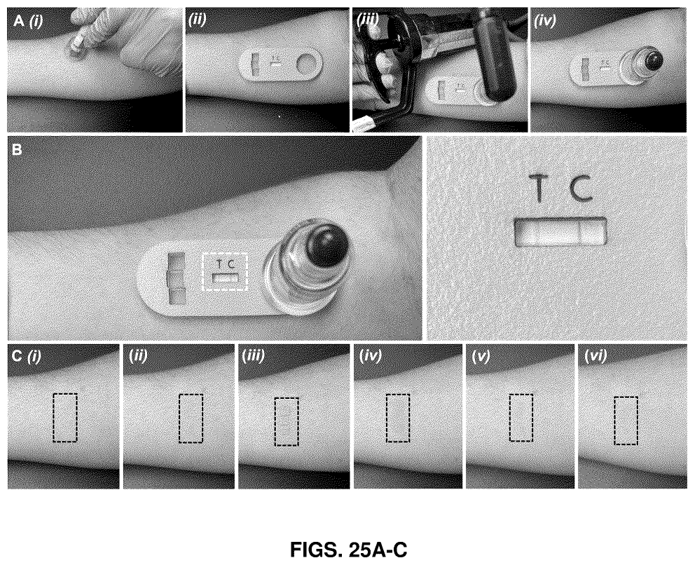

Fluid flow through the skin patch. The flow characteristics of liquid within the patch were first evaluated using an artificial skin model. For this experiment, the bandage tape was removed from the patch to facilitate visualization of fluid flow through the microchannels and LFIA test strip. Within 2 min of applying suction to the patch, liquid was extracted from the micropores (FIG. 25A, ii). The extracted liquid flowed into the patch (via the sampling ports) and through the microchannels (FIG. 25, iii). Upon encountering the LFIA test strip, the liquid was wicked through the strip via surface tension (FIG. 25A, iv). As the liquid moved across the conjugate pad, AuNP-anti-human IgG conjugates were reconstituted and transported to the test and control lines. By maintaining constant vacuum pressure, fluid was continuously extracted from the skin, which served to wash away unbound AuNP-anti-human IgG conjugates from the test strip. After ˜11 min, the strip was fully wetted by the liquid and unbound AuNP-anti-human IgG conjugates were completely removed from the strip, resulting in a negligible background signal (FIG. 25B). Further experimentation was performed to evaluate the extraction of dermal ISF from human skin and its transport through the skin patch (FIG. 27). Similar flow characteristics were observed where dermal ISF could be quickly extracted and transported through the LFIA test strip within 18 min. The inventors attribute the slightly longer time required for full wetting of the test strip and removal of unbound AuNP-anti-human IgG conjugates in human skin compared with the artificial skin model to the higher viscosity of ISF.

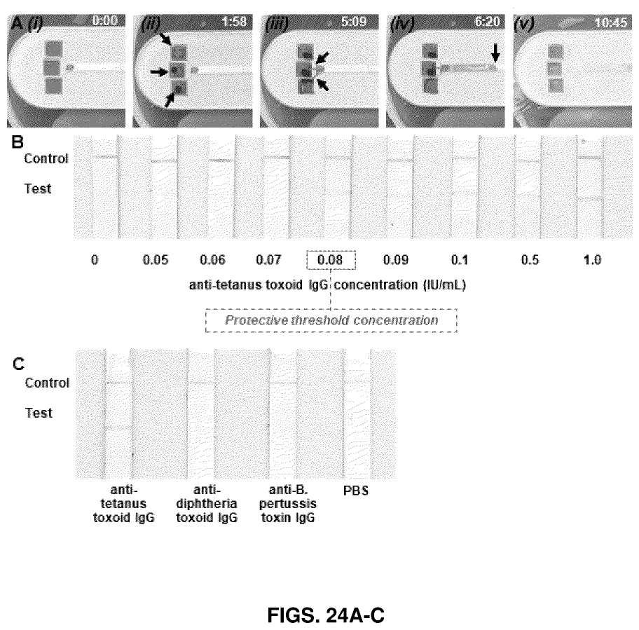

Sensitivity and selectivity of the tetanus lateral flow assay. The inventors first assessed the detection sensitivity of the assay using ISF simulant spiked with varying concentrations of anti-tetanus toxoid IgG. A test line was generated for samples containing anti-tetanus toxoid IgG at concentrations≥0.08 IU/mL, where the intensity of line was correlated with the antibody concentration (FIG. 24B). Several assay parameters, including the AuNP concentration and antibody concentrations, were optimized so that a test line was generated only when the anti-tetanus toxoid IgG concentration in the sample was ≥0.08 IU/mL, which was used as the protective threshold concentration for ISF, indicating immunity against tetanus infection. This threshold concentration was determined by applying the ratio of anti-tetanus toxoid IgG levels in ISF to blood (0.8) (FIG. 23) to the established protective threshold concentration of anti-tetanus toxoid IgG in blood (0.1 IU/mL) (Tiwari et al., 2021; Hanvatananukul et al., 2020; Broder et al., 2006). No test line was generated for samples containing anti-tetanus toxoid IgG at concentrations<0.08 IU/mL. All the samples generated a dark control line, validating the test results.

The analytical specificity of the assay was evaluated by testing ISF samples containing anti-tetanus toxoid IgG, anti-Bordetella pertussis toxoid IgG or anti-diphtheria toxoid IgG. Vaccination for diphtheria, pertussis and tetanus is commonly administered as a single dose (Tdap) (Havers et al., 2020); therefore, antibodies to diphtheria and pertussis toxoids were selected for specificity testing due to their potential to interfere with the tetanus toxoid antigen (Kadam et al., 2019). Measurement of a phosphate-buffered saline (PBS) sample was performed and used as a blank control. As shown in FIG. 24C, only the sample containing anti-tetanus toxoid IgG generated a test and control line, indicating a positive test result. In contrast, only the control line was generated for the samples containing irrelevant antibodies, which was identical to the PBS sample, indicating a negative test result. These results demonstrate that this assay is highly specific to anti-tetanus toxoid IgG and exhibits negligible cross-reactivity with potentially interfering antibodies.

In situ detection of anti-tetanus toxoid IgG using the skin patch. To evaluate the functionality of the patch for in situ protein detection, the inventors tested it on a volunteer. To initiate the test, the MN array was first applied to the anterior forearm using an MN applicator (FIG. 25A, i). The patch was adhered to the skin in a manner such that the sampling ports were aligned with the MN insertion sites (FIG. 25A, ii). Next, a vacuum cup was attached to the vacuum port of the patch and vacuum pressure was generated using a hand pump (FIG. 25A, iii). The pump was detached from the cup and vacuum was maintained until ISF was wicked through the entirety of the test strip (FIG. 25A, iv). The test results were observable within 18 min of vacuum application (FIG. 25B). Both the test line and control line were generated, indicating an anti-tetanus toxoid IgG concentration of ≥0.08 IU/mL, which is consistent with the IgG concentration that was measured in the dermal ISF of this individual (Participant ID 1, FIG. 23) using ELISA.

The inventors also investigated whether the use of the patch or testing procedure caused any adverse effects to the skin. MN insertion resulted in slight redness at the MN application sites (FIG. 25C, ii). Skin redness and mild swelling localized at the MN insertion sites was observed as a result of suction being applied to the skin (FIG. 25C, iii-iv), however, these reactions are common and benign effects associated with vacuum/cupping therapy (Dalton & Velasquez, 2017). Within six hr, swelling had completely subsided, and only very faint redness remained (FIG. 25C, v). No other reactions were observed, and the redness completed resolved within 24 hr after testing (FIG. 25C, vi). Overall, testing resulted in very minor adverse effects that were quickly resolved.

### Example 6—Discussion

Rapid diagnostic testing is used for various applications, including the detection of current or past infections, monitoring disease progression or therapeutic response, and determining immune status to guide vaccination decisions. RDTs enable such testing to be performed outside of laboratory settings by individuals with minimal or no training. Due to their low-cost, quick turnaround time and ease of use, RDTs are widely used throughout the world, particularly in resource-limited settings that lack basic infrastructure and medical resources. However, these tests commonly rely on blood sampling, which poses risks of infection, can lead to complications in infants and individuals with blood disorders, and can deter individuals with blood or needle phobias from getting tested. To address these challenges, this skin patch offers blood-free detection of protein biomarkers in ISF, which can be sampled from the skin in a minimally invasive and nearly painless manner. MN-based biosensing platforms for in situ ISF extraction and analyte detection have previously been reported (Zhu et al., 2023; Zheng et al., 2022; Friedel et al., 2023b; Ribet et al., 2018; Freeman et al., 2023; De la Paz et al., 2023; De la Paz et al., 2021); however, they required the use of bulky and/or specialized electronic components, such as electrochemical analyzers or custom circuits, and were limited to the detection of small molecules (e.g., metabolites, drugs). In the inventors' approach, a MN-based ISF sampling technique is combined with a colloid gold-based LFIA, and vacuum-assisted extraction system integrated on a microfluidic skin patch, enabling rapid in situ detection of protein biomarkers in dermal ISF. This device does not require any sample processing (e.g., centrifugation, purification, dilution), resulting in a simplified testing protocol and a reduced risk of disease transmission due to sample handling. Furthermore, the colorimetric readout enables the test results to be observed by the naked eye without requiring specialized instrumentation. Unlike previously reported ISF sampling techniques that rely on specialized equipment or electric vacuum pumps, this device uses an inexpensive (<$10) vacuum cup and hand pump commonly used in cupping therapy, making it portable and amenable for use in both clinical and point-of-care settings.

A major limitation of existing MN-based ISF sampling techniques is that the collected fluid volumes are too low (1-6 μL) (Ribet et al., 2023; Kim et al., 2021; Samant et al., 2020) for biomolecular analysis using LFIAs, which require at least ˜15 μL of sample for testing. One of the key advantages of this device is its ability to extract larger (>15 μL) amounts of dermal ISF within a short period, which was achieved by implementing several strategies. First, a high-density MN array is used to generate hundreds of micropores in the skin, providing multiple paths for ISF extraction. A major challenge associated with vacuum assisted ISF sampling is that human skin is highly elastic and easily deforms when vacuum pressure is applied, causing the micropores to close. To overcome this challenge, the microfluidic network is fabricated using a semi-rigid PMMA substrate, which keeps the skin taut when suction is applied and induces the opening of the micropores, facilitating ISF extraction. The inventors observed that fabricating the microfluidic network from thinner/less-rigid PMMA caused the skin to deform significantly when suction was applied to the patch, resulting in no ISF extraction. Additionally, the adhesive backing of the patch creates an air-tight seal with the skin, enabling vacuum pressure to be maintained throughout the test. Combining the use of the vacuum cup with the skin patch to generate suction resulted in the creation of a large pressure gradient across the skin, driving the flow of ISF through the micropores. While applying suction to the skin resulted in minor adverse effects (e.g., slight redness), these effects completely resolved within 24 hr. Compared to the adverse reactions and complications that can occur with blood sampling (e.g., pain, bruising, hematoma and thromboembolism) (Heinemann, 2008; Robb, 1985), this test is significantly less invasive and safer, which will make it more readily accepted by individuals with blood or needle phobias.

Measurements of IgG antibodies has clinical relevance in determining an individual's immunity to specific pathogens and guiding vaccination decisions. The ability to determine an individual's immunity to diseases in a rapid and minimally invasive manner is particularly valuable for individuals who are under-vaccinated or unaware of their vaccination status, putting them at an elevated risk of infection (Causey et al., 2021). In this work, anti-tetanus toxoid IgG was used as the target biomarker to demonstrate proof-of-principle of this technology. Using a MN- and vacuum-assisted sampling technique, ISF was successfully collected from human volunteers. ELISA measurements of ISF and blood samples revealed the presence of anti-tetanus toxoid antibodies in both fluids, where concentrations in ISF were correlated with those in blood. This result is significant since it validates the diagnostic utility of dermal ISF for the detection of disease-specific immune antibodies and protein biomarkers. The functionality of the skin patch was further demonstrated through rapid ISF extraction from human skin and generation of test results in <20 min, showcasing its potential for rapid diagnostic testing.

The inventors envision that this device can be readily modified to be used for determining immunity to other diseases, such as diphtheria and pertussis, by replacing the tetanus toxoid protein with a different capture antigen and modifying the assay parameters to adjust the protective threshold concentration. Alternatively, modifications can be made to the assay enabling the detection of other protein biomarkers associated with viral, parasitic, and bacterial infections, such as HIV infection, malaria, Dengue fever or Lyme disease, further expanding the utility of this device for diagnostic testing.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.
Al-Kasasbeh et al., Drug Deliv. Transl. Res. 10, 690-705. 10.1007/s13346-020-00727-2. (2020).
Aukland & Nicolaysen, Physiol. Rev. 61, 556-643. 10.1152/physrev.1981.61.3.556. (1981).
Baker, Sport. Med. 47, 111-128. 10.1007/s40279-017-0691-5. (2017).
Bogers et al., Eur. Neuropsychopharmacol. 25, 319-324 (2015).
Broder et al., MMWR. Recomm. Rep. 55, 1-34 (2006).
Causey, K. et al., Lancet 398, 522-534 (2021).
Chen & Lee, Micromachines 12, (2021).
Dalton & Velasquez, Public Heal.—Open J. 2, 59-63 (2017).
Davis et al., J. Biomech. 37, 1155-1163. 10.1016/j.jbiomech.2003.12.010. (2004).
De la Paz et al., Talanta 254, (2023).
De la Paz et al., Anal. Chem. 93, 12767-12775 (2021).
Dingerdissen et al., JCO Clin. Cancer Informatics, 210-220 (2020).
Dutkiewicz & Urban, Philos. Trans. R. Soc. A Math. Phys. Eng. Sci. 374. 10.1098/rsta.2015.0380. (2016).
Freeman et al., ACS Sensors 8, 1639-1647 (2023).
Friedel et al., Nat. Biomed. Eng. (2023a).
Friedel et al., Lab Chip 23, 3289-3299 (2023b).
Grady et al., J. Diabetes Sci. Technol. 8, 691-698. 10.1177/1932296814533172. (2014).
Ghezzi et al., J. Dent. Res. 79, 1874-1878. 10.1177/00220345000790111001. (2000).
Hanvatananukul et al., Int. J. Infect. Dis. 96, 422-430 (2020).
Havers et al., MMWR. Morb. Mortal. Wkly. Rep. 69, 77-83 (2020).
Heinemann, J. Diabetes Sci. Technol. 2, 919-921 (2008).
Jina et al., J. Diabetes Sci. Technol. 8, 483-487. 10.1177/1932296814526191. (2014).
Kalra & Lowe, A. J. Mater. Sci. Eng. 5. 10.4172/2169-0022.1000254 (2016).
Kadam et al., Methods 158, 33-43 (2019).
Kim et al., Sci. Rep. 11, 1-11 (2021).
Kim et al., J. Mech. Behav. Biomed. Mater. 78, 480-490. 10.1016/j.jmbbm.2017.12.006. (2018)
Kolluru et al., Adv. Healthc. Mater. 8, 1-19. 10.1002/adhm.201801262. (2019).
Kool et al., Proteomics 7, 3638-3650 (2007).
Kuppusami & Oskouei, Univers. J. Biomed. Eng. 3, 9-14 (2015).
Krogstad et al., Br. J. Dermatol. 134, 1005-1012. 10.1111/j.1365-2133.1996.tb07934.x. (1996).
Larrañeta et al., Int. J. Pharm. 472, 65-73 (2014).
Lassandro et al., 13, (2021).
Leeman et al., L. Anal. Bioanal. Chem. 410, 4867-4873. 10.1007/s00216-018-1127-2 (2018).
Makvandi, P. et al. Engineering Microneedle Patches for Improved Penetration: Analysis, Skin Models and Factors Affecting Needle Insertion. Nano-Micro Letters vol. 13 (Springer Singapore, 2021).
Mccrudden et al., Exp. Dermatol. 24, 561-566. 10.1111/exd.12723. (2015).
Miller et al., Commun. Biol. 1, (2018).
Mukerjee et al., A Phys. 114, 267-275. 10.1016/j.sna.2003.11.008. (2004).
Müller et al. J., Proteome Res. 11, 3715-3727 (2012).
Parolo et al., Nat. Protoc. 15, 3788-3816 (2020).
Pieber et al., 0585, 401-408. (2008).
Piorino et al., Curr. Opin. Biotechnol. 76, 102738 (2022).
Reed & Rubin, 10.1093/cvr/cvql43 10.1093/cvr/cvql43. (2010).
Ribet et al., Proc. IEEE Int. Conf Micro Electro Mech. Syst. 2018-Janua, 408-411 (2018).
Ribet et al., Sens. Bio-Sensing Res. (2020).
Ribet et al., Adv. Healthc. Mater. 2202564, 1-11 (2023).
Robb, IRB Ethics Hum. Res. 7, 7-9 (1985).
Rozenfeld & Kalichman, J. Bodyw. Mov. Ther. 20, 173-178. 10.1016/j.jbmt.2015.11.009. (2016).
Saifullah & Faraji Rad, Adv. Mater. Interfaces 10, (2023).
Samant & Prausnitz, Proc. Natl. Acad. Sci. U.S.A 115, 4583-4588 (2018).
Samant et al., Sci. Transl. Med. 12, 1-16 (2020).
Schmook et al., Int. J. Pharm. 215, 51-56 (2001).
Sloop et al., 10.1016/s0022-2275(20)38701-0 10.1016/s0022-2275(20)38701-0 (1987).
Sieg et al., Clin. Chem. 50, 1383-1390. 10.1373/clinchem.2004.032862. (2004).
Sim et al. Adv. Sci. 9, 1-29 (2022).
Svedman et al., BMC Dermatology Plasma proteins in a standardised skin mini-erosion (I): permeability changes as a function of time. (2002).
Tran et al., J. Proteome Res. 17, 479-485 (2018).
Tiwari et al., Centers Dis. Control Prev. 315-328 (2021).
Turewicz et al., Adv. 1, 1-3 (2021)Venugopal et al., IEEE Sens. J. 8, 71-80. 10.1109/JSEN.2007.912544. (2008).
Waghule et al., Biomed. Pharmacother. 109, 1249-1258 (2019).
Xu et al., Advances of Microneedles in Biomedical Applications. Molecules (2021).
Youl et al., Proteomics 5, 290-296. 10.1002/pmic.200400889. (2005).
Zheng et al., 2108607, 1-11 (2022).
Zhu et al., ACS Appl. Mater. Interfaces (2023) doi:10.1021/acsami.3c00573.

1. A transdermal microneedle array (TMNA) comprising a first microneedle (MN) array composed of a plurality of solid microneedles (MNs).

2. The TMNA of claim 1, wherein the MN array comprises about 25 to about 1000 MNs, about 100 to about 400 MNs or about 100 to about 200 MNs.

3. The TMNA of claim 2, wherein the MNs are arranged in a 5×5, 10×10, 15×15, 20×20, or 30×30 configuration.

4. The TMNA of claim 1, wherein the MN array is formed using a 3D-printing/stereolithography, such as using a master mold, such as one made from PDMS, and/or the MN array is made of an epoxy-based photoresist material (e.g., SU-8) or from a ceramic or metallic material.

5. The TMNA of claim 1, wherein the MN array is coated with a biocompatible material.

6. The TMNA of claim 5, wherein the biocompatible material is chitosan, polyethylene glycol or parylene.

7. The TMNA of claim 1, wherein the MN array microneedles are about 250 m or 300 m to about 1000 m, about 450 m to about 750 m, or about 600 m in height, and/or wherein the MN array is about 7.5 mm×7.5 mm, about 10 mm×10 mm, or about 7.5-10 mm×7.5-10 mm, and/or wherein the needle spacing in said MN array is about 300 μm to about 500 μm, or about 400 μm, and/or wherein the MN array is a 20×20 array with a needle length of about 450 μm.

8-10. (canceled)

11. A kit comprising the TMNA of claim 1 and a rigid skin patch with one or more cut out configured to fit the TMNA.

12. The kit of claim 11, wherein the skin patch is fabricated from a plastic substrate coated on at least one side with an adhesive.

13. The kit of claim 11, further comprising a lateral flow test strip and a vacuum port in operable connection lateral flow test strip.

14. The kit of claim 13, wherein the lateral flow test strip comprises a membrane, such as a nitrocellulose or cellulose membrane, wherein the lateral flow test strip is connected to the patch by microchannels, such as channel of about 100-500 um width, or about 200 m width.

15. The kit of claim 14, the lateral flow test strip further comprises an antigen binding agent disposed in or on said membrane.

16. A method of obtaining interstitial fluid (ISF) from a subject using the kit of claim 11, the method comprising:
(a) applying the skin patch to a subject;
(b) contacting the subject with the TMNA at the cut out and removing the TMNA thereafter;
(c) affixing the skin patch to the skin over the TMNA insertion site;
(d) applying suction to the skin at the area of the cut out; and
(e) collecting the ISF removed from the subject by the suction.

17. The method of claim 16, wherein step (b) comprises multiple applications of the TMNA to the subject at the cutout, such as 2, 3, 4, 5 or more times.

18. The method of claim 16, wherein the application or applications of the TMNA is performed with a spring-loaded applicator or manually by hand.

19. The method of claim 16, wherein the skin patch comprises multiple cut outs, such as 2, 3, 4, 5 or more cut outs.

20. The method of claim 16, wherein the suction is generated by attaching a hand operated pump or an automated and/or electric pump, such as one generating about 20 to 60 kPa, or about 44 kPa, such as using vacuum cup diameter of 34 mm and pressure of 44 kPa, with MN array of 2×2 square matrix and 3 rectangles in a row.

21. The method of claim 16, wherein suction is applied for about 5-30 min, such as about 5, about 10, about 15, about 20, about 25 or about 30 min, and/or wherein about 10 μl, 15 μl, 20 μl, about 25 μl, about 30 μl, about 35 μl, about 40 μl, about 45 μl, about 50 μl, about 60 μl, or about 65 μl of ISF are obtained, and/or wherein the MN array is a 20×20 array with a needle length of about 450 μm, the number of MN applications is three, the pressure is 44 kPa, and the duration of step (d) is about 20 min.

22-23. (canceled)

24. The method of claim 16, further comprising analyzing one or more components of collected ISF, such as by detecting their presence by the lateral flow assay.

25. A method of obtaining interstitial fluid (ISF) from a subject using the kit of claim 11, the method comprising:
(a) applying the skin patch to a subject;
(b) contacting the subject with the TMNA at the cut out and removing the TMNA thereafter;
(c) applying suction to the skin at the area of the cut out; and
(d) collecting the ISF removed from the subject by the suction,
optionally further comprising analyzing one or more components of collected ISF.
